(19) United States
(12) Patent Application Publication
(10) Pub. No.: US 2022/0204802 A1
(43) Pub. Date: Jun. 30, 2022

(54) HYDROGELS HAVING TUNABLE CROSS-LINKING DENSITIES AND REVERSIBLE PHASE TRANSITIONS AND METHODS FOR THEIR USE

(71) Applicant: The Regents of the University of California, Oakland, CA (US)

(72) Inventors: Huinan Liu, Riverside, CA (US); Changlu Xu, Hacienda Heights, CA (US)

(21) Appl. No.: 17/565,201

(22) Filed: Dec. 29, 2021

Related U.S. Application Data
(60) Provisional application No. 63/132,327, filed on Dec. 30, 2020.

(51) Int. Cl. C09D 105/08; B33Y 70/00; B29C 64/314; A61L 27/20; B29C 64/118; B33Y 40/10; A61L 27/52; C08J 3/075; C08J 3/24; B33Y 10/00; C08B 37/08; B29K 105/00

(52) U.S. Cl. CPC C09D 105/08; B33Y 70/00; B29C 64/314; A61L 27/20; B29C 64/118; B33Y 40/10; B29K 2105/0061; C08J 3/075; C08J 3/24; B33Y 10/00; C08B 37/0072; C08J 2305/08; A61L 27/52

(57) ABSTRACT

Provided is a method that achieves tunable crosslinking and reversible phase transition of hydrogels. The method is useful for preparing 3D-printable hydrogel, for example, for wound healing, aneurysm treatment or tissue regeneration.

### PRIORITY

This application claims priority to U.S. Provisional Application No. 63/132,327, filed 30 Dec. 2020. The entire content of this United States Provisional Application is hereby incorporated herein by reference.

### BACKGROUND

Hydrogels are crosslinked three-dimensional (3D) polymeric networks that can maintain high water content in their structures and can be synthesized from both synthetic and natural polymers. Currently there is a need for hydrogels that provide tunable crosslinking and reversible phase transition, and that are suitable for 3D printing applications.

### SUMMARY

One embodiment provides, a hydrogel having tunable crosslinking density and reversible phase transition that is suitable as an ink for three-dimensional (3D) printing.

One embodiment provides, a 3D printable aqueous ink that comprises a hydrogel as described herein.

One embodiment provides, a method for 3D printing a three-dimensional object, the method comprising, providing a 3D ink as described herein; and 3D printing the three-dimensional object using the 3D ink as a feedstock.

One embodiment provides a wound healing device that comprises a hydrogel as described herein.

One embodiment provides a scaffold for cartilage that comprises a hydrogel as described herein.

One embodiment provides an aneurysm treatment device that comprises a hydrogel as described herein.

One embodiment provides a scaffold for tissue regeneration that comprises a hydrogel as described herein.

One embodiment provides a method for preparing a 3D printable ink comprising, combining hyaluronic acid and Fe³⁺ ions and adjusting the concentration of Fe⁺ or H⁺ to provide the 3D printable ink. In one embodiment, the 3D printable ink comprises a hydrogel that comprises hyaluronic acid and Fe³⁺ ions.

The hydrogels described herein achieve crosslinking, reversible phase transition, and 3DP capability based on dynamic carboxylate-metallic ion coordination of innate carboxylic groups and metal (e.g. Fe³⁺) ions. Ion concentration, pH, and reaction time all impact coordination states and crosslinking densities of the hydrogels. Three types of liquid-to-solid hydrogels (HyA_L, HyA_M, and HyA_H) have been prepared. The dense stiff solid HyA_H had a much lower porosity in microstructure than HyA_M, and thus exhibited a dramatic improvement in tensile strength and tensile modulus. Additionally, two 3DP strategies (a cold-stage method and a direct writing method) have been carried out using the hydrogels, based on tunable crosslinking densities and reversible phase transition capability.

The invention also provides processes and intermediates disclosed herein that are useful for preparing the hydrogels described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C. Schematic for reversible coordination state of crosslinked HyA hydrogels. (A) Illustration for HyA molecule mixed with FeCl₃ solution. Left graph shows there is one carboxyl group in each disaccharide unit of HyA molecule chain. The FeCl₃ solution consists of Fe³⁺, complex of Fe³⁺, H⁺, and other Cl⁻ counter ions (Middle). Fe³⁺ could form a mixture of mono-, bi-, and tridentate with carboxyl groups on HyA chains, and the carboxyl groups also can interact with H⁺ in the acidic Fe³⁺ solution (Right). (B) Schematic diagram shows Fe³⁺ and H⁺ concentration determine final coordination state of crosslinked HyA hydrogels, and the time will affect the intermediate coordination state of crosslinked HyA hydrogels. (C) Schematic diagram shows three types of reversible crosslinked HyA hydrogels with varied Fe³⁺-carboxyl coordination. (1) HyA_L in which H⁺ replace the Fe³⁺ in the coordination site, resulting in the low degree of crosslinking. (2) HyA_M has a mixture of mono-, bi-, or tridentate coordination, leading to a medium degree of crosslinking. (3) HyA_H mainly has tridentate coordination and shows the high crosslinking density.

FIGS. 2A-2D. The structural stability and storage modulus of crosslinked HyA hydrogels demonstrated the effects of Fe³⁺ concentrations and pH values on the reversible phase transition of crosslinked HyA hydrogels. (A) The representative photographs of as-injected (or as-printed) network pattern of HyA in the well at time zero (T=0 h) and crosslinked HyA gels after being immersed in 2 to 300 mM of FeCl₃ solutions with innate pH values for 24 h (T=24 h). (B) The representative photographs of as-injected (or as-printed) network pattern of HyA in the well at time zero (T=0 h) and crosslinked HyA gels after being immersed in 10 mM FeCl₃ solution with a decreased pH value of 2.4, 2.2, and 2.15, respectively; in 20 mM FeCl₃ solution with a decreased pH value of 2.22, 2.15, and 2.1, respectively; and in 30 mM FeCl₃ solution with a decreased pH value of 2.11 and 2.07, respectively. (C) The curves of storage modulus-shear strain for HyA hydrogels cross linked by 10 mM, 20 mM, and 30 mM FeCl₃ solutions with a constant pH of 1.7 for 5 min (Left), 15 min (Middle), and 30 min (Right). (D) The curves of storage modulus-shear strain for HyA hydrogels cross linked in the 20 mM FeCl₃ solution with an adjusted pH value of 1.5, 1.7, and 1.9, respectively, for 5 min (Left), 15 min (Middle), and 30 min (Right).

FIGS. 3A-3G. Reversible phase transition of crosslinked HyA hydrogels with medium (HyA_M), low (HyA_L) and high (HyA_H) crosslinking density via dynamic metal-ligand coordination. (A) Evidence of phase transition from HyA_M to HyA_L, HyA_M to HyA_H, and HyA_L to HyA_H. Specifically, HyA dissolved in DI water and formed a uniform solution layer (Light blue) in the PTFE mold. The coordination states of crosslinked HyA hydrogels dynamically changed over time after the addition of FeCl₃ solution. After adding FeCl₃ solution to HyA solution for 5 minutes, a soft flexible gel (named HyA_M) formed; after adding FeCl₃ solution to HyA solution for 1 hour, a viscous liquid gel (named HyA_L) formed. HyA_M bent when being held vertically with a tweezer. When immersing HyA_M or HyA_L in DI water for 24 hours, a hard and stiff gel (named HyA_H) formed and HyA_H did not bend when being held vertically with a tweezer. (B) Evidence of phase transition from HyA_L to HyA_M. HyA_L is an injectable viscous liquid, useful for direct writing or direct printing; and the as-injected HyA_L transformed to HyA_M after 30 mM of FeCl₃ solution was added onto it for 5 minutes. (C) Evidence of phase transition from HyA_H to HyA_M and HyA_H to HyA_L. HyA_H transformed to HyA_M after being immersed in 300 mM FeCl₃ solution for 1 hour; HyA_H transformed to HyA_L after 2-h immersion in 300 mM FeCl₃ solution. (D) The kinetics for the phase transition of HyA_M to HyA_H. HyA_M in DI water showed a dramatic volume shrinkage over time to become HyA_H (from left to right). (E) Diameter and volume change ratio over time during the phase transition from HyA_M to HyA_H in DI water. Diameter is shown on the left axis with data in red and volume change ratio is shown on the right axis with data in blue. Data are shown as mean±standard deviation (n=3). Inset is the magnified graph of the first 4 hours. (F) Water content of the gel over time during the phase transition from HyA_M to HyA_H in DI water. Data are shown as mean±standard deviation (n=3). Inset is the magnified graph of the first 4 hours. (G) Cumulative Fe³⁺ released over time during the phase transition from HyA_M to HyA_H in DI water. Data are shown as mean±standard deviation (n=3). Inset is the magnified graph of the first 4 hours.

FIGS. 4A-4D. Microstructures of crosslinked HyA hydrogels and thermal properties. (A) SEM images of cross-sections of lyophilized HyA_M, HyA_H, and HyA at the original magnifications of 1000× and 10000×, and the SEM/EDS maps of HyA_M and HyA_H at the original magnification of 10000× The large pores in left were artifacts from lyophilization process. Red circles indicate the microstructures that were magnified on the bottom.

Microstructural porosity of HyA_M, HyA_H, and HyA were 82.88% 1.52%, 26.98% 3.31%, and 86.78% 1.9%, respectively. Red dots in right images indicated Fe element. Atomic percentage of Fe element in HyA_M and HyA_H were 3.3% and 2.6%, respectively. (B) Mass (%) of HyA_L, HyA_M, HyA_H, and HyA as the temperature increased in the TGA testing. The decomposition temperature of HyA_L was 150° C., HyA_M was 202° C., HyA_H was 205, and HyA was 230° C. (C) DSC curves of HyA_L, HyA_M, HyA_H, and HyA. (D) The Tg for HyA_L was 125° C., HyA_M was 142° C., HyA_H was 150° C., and HyA was 140° C.

FIGS. 5A-5H. Mechanical properties of crosslinked HyA hydrogels. (A) The photographs of HyA_M elongation during the tensile testing. (B) The photographs of HyA_H elongation during the tensile testing. (C) The representative stress-strain curves of HyA_M (orange curve with data shown on the left orange axis) and HyA_H (wine curve with data shown on the right wine axis). (D) Tensile strength of HyA_M (orange bar with data shown on the left orange axis) and HyA_H (wine bar with data shown on the right wine axis). Data are shown as mean±standard deviation (n=3). (E) Tensile modulus of HyA_M (orange bar with data shown on the left orange axis) and HyA_H (wine bar with data shown on the right wine axis). Data are shown as mean±standard deviation (n=3). (F) Elongation of HyA_M (orange bar) and HyA_H (wine bar) at break in the tensile testing. Data are shown as mean standard deviation (n=3). (G) Storage modulus (G′) and loss modulus (G″)-shear strain curves of HyA_L (yellow) and HyA (dark) from the rheological testing. (H) The curves of viscosity over shear rate for HyA_L (yellow) and HyA (dark).

FIGS. 6A-6H. The conditions required for 3D-printing of crosslinked HyA gels and comparisons of two different methods for creating 3D-printed crosslinked HyA hydrogels. (A) Boundary conditions of FeCl₃ concentrations and pH values for crosslinked HyA gel to be printable. Green region indicates the conditions with feasible FeCl₃ concentration and pH value for achieving printability of crosslinked HyA gels. (B) Storage modulus (G′) and loss modulus (G″)-shear strain curves of crosslinked HyA gels prepared by immersing HyA into 300 mM FeCl₃ solution for 5 minutes (pink square), 15 minutes (green circle), 30 minutes (light magenta diamond), and 120 minutes (dark yellow triangle), respectively, and HyA control (dark pentagram). (C) Viscosity curves of crosslinked HyA gels prepared by immersing HyA into 300 mM FeCl₃ solution for 5 minutes (pink square), 15 minutes (green circle), 30 minutes (light magenta diamond), and 120 minutes (dark yellow triangle), respectively, and HyA control (dark pentagram). (D) Illustration for 3D-printing of partially cross-linked HyA hydrogels (named as printable HyA gels, HyA_P) using the cold-stage method. That is, HyA_P was printed on a cold stage of 0° C., and the as-printed hydrogel was then immersed in DI water to transform to HyA_H. The HyA_P (left) was prepared by immersing HyA in 300 mM FeCl₃ solution for 15˜30 min and exhibited different states of mono- and bidentate coordination and transformed to HyA_H (right) with tridentate coordination after immersion in DI water. (E) Illustration for 3D-printing of HyA_P using the direct writing method, in which The HyA-P was directly printed in DI water at room temperature. The HyA_P (left) was prepared by immersing HyA in 300 mM FeCl₃ solution for 15˜120 minutes. (F) Optical micrographs of crosslinked HyA hydrogels that were printed on cold stage and then immersed in DI water for 24 hours, with both top view (Top) and bottom view (Bottom) of the 3D-printed hydrogels. The HyA_P was obtained by immersing the HyA solution in 300 mM FeCl₃ solution with a pH value of 1.3 for 15 minutes. (G) Optical micrographs of crosslinked HyA hydrogels directly printed in DI water and immersed for 24 hours, with both top view (Top) and bottom view (Bottom) of the 3D-printed hydrogels. The HyA_P was obtained using 300 mM FeCl₃ solution with a pH value of 1.3 for 1 hour. (H) Structural stability of the 3D printed crosslinked HyA gels using the cold-stage method and direct writing method. Representative optical micrographs and aspect ratio of the cross-sections of filaments printed via the cold-stage method (grey) and direct writing method (green). Filaments printed by the cold-stage method were waited for 0 minutes, 1 minute, 5 minutes, and 10 minutes, respectively in air after printing and immersed in DI water for 24 hours, and filaments printed by direct writing method were immersed for 24 hours. The aspect ratio is height/width (AR=H/W). Data are mean standard deviation (n=5).

FIGS. 7A-7B. Demonstration of cytocompatibility of HyA hydrogels in BMSC culture. (A) Representative fluorescence images of BMSCs directly in contact with the samples (direct contact) and cells on the culture plate surrounding each corresponding sample (indirect contact) after 24-hour direct exposure culture. Scale bar=100 μm for all images. (B) Cell adhesion densities under direct and indirect contact conditions in the groups of HyA_H_3D, HyA_H, HyA_M, HyA_P, HyA_L, and the control groups of Glass, Fe@2.09 mM, Fe@6.08 mM, HyA, and Cell. Data are shown as mean±standard deviation (n=3); *p<0.05, **p<0.01, ***p<0.001.

FIGS. 8A-8D. Demonstration of HyA degradation and associated changes in culture media via media analysis of pH value and concentrations of Fe³⁺ and Ca²⁺. (A) pH values of media upon adding samples before culture. (B) pH values of post-culture media. (C) Fe³⁺ concentrations in post-culture media. (D) Ca²⁺ concentrations in post-culture media. Data are shown as mean±standard deviation (n=3); *p<0.05, **p<0.01, ***p<0.001.

FIG. 9. The innate pH values of FeCl₃ solutions at different concentrations.

FIGS. 10A-10D. Tensile testing results of HyA_H at different strain rates. (A) Representative stress-strain curves from the tensile testing of HyA_H at different strain rates of 10 mm/min, 20 mm/min, and 30 mm/min. (B) Tensile strength of HyA_H at different strain rates of 10 mm/min, 20 mm/min, and 30 mm/min. (C) Tensile modulus of HyA_H at different strain rates of 10 mm/min, 20 mm/min, and 30 mm/min. (D) Elongation at break of HyA_H at different strain rates of 10 mm/min, 20 mm/min, and 30 mm/min. Data in (B, C, D) are shown as mean±standard deviation (n=3).

FIGS. 11A-11B. Structural stability of (A) the printable gel (HyA_P) with a crosslinking density between HyA_L and HyA_M over a period of 20 minutes, in contrast to the rapid structural change of (B) the HyA control over 15 seconds, after they were printed or injected onto the surface of a petri dish.

FIG. 12. Optical image of the cells adhered on the culture plate before adding any samples, that is, the cells at time zero (T=0).

FIG. 13. Representative optical images of BMSCs directly in contact with the samples (direct contact) and cells on the culture plate surrounding each corresponding sample (indirect contact) after 24-hour direct exposure culture. Scale bar=100 μm for all images.

FIG. 14. Photographs of the samples in the culture media before (T=0) and after (T=24) the prescribed 24-h incubation. The dashed circles indicate the samples in the media. The brightness and contrast of the images were adjusted to highlight the samples in the media. Scale bar=5 mm for all images.

FIG. 15. Photographs of the collected media containing visible degradation products from the samples. Dashed circles are used to indicate the degradation products. Scale bar=5 mm for all images.

FIGS. 16A-16E. Rheological results showing the structural stability of 5 w/v % HyA gels at different HCl concentrations of 10, 30, 50, and 80 mM. (A and B) Storage modulus (G′) and loss modulus (G″) of HyA gels at (A) different frequencies and (C) shear strains. (C and D) The ratio of G′/G″ for HyA gels at (C) different frequencies and (D) shear strains. (E) The viscosity of HyA gels at different shear rates.

FIG. 17. Photographs showing the adhesion between the crossed hydrogel filaments.

The crossed filaments were prepared by injecting 5 w/v % HyA solution with 10-50 mM HCl concentrations in 30 mM FeCl₃ solution and reacting for 10 s.

FIGS. 18A-18C. Rheological results showing the structural stability of 5 w/v % HyA gels at different Ca²⁺ concentrations of 0.5, 1, 2, and 2.5 M. (A) Storage modulus (G′) and loss modulus (G″) of HyA gels at different shear strains. (B) The ratio of G′/G″ for HyA gels at different shear strains. (C) The viscosity of HyA gels at different shear rates.

FIG. 19. Optical micrographs of crosslinked HyA hydrogels that were printed on cold stage and then immersed in 50 mM FeCl₃ solution followed by immersing in DI water for 24 hours, with both top view (Top) and bottom view (Bottom) of the 3D-printed hydrogels. The HyA_P was 5 w/v % HyA solutions with 2.5 M Ca²⁺.

FIGS. 20A-20E. Rheological results showing the structural stability of 5 w/v % HyA gels at 2 M Ca²⁺ and different HCl concentrations of 10, 20, and 30 mM. (A and B) Storage modulus (G′) and loss modulus (G″) of HyA gels at (A) different frequencies and (B) shear strains. (C and D) The ratio of G′/G″ for HyA gels at (C) different frequencies and (D) shear strains. (E) The viscosity of HyA gels at different shear rates.

FIG. 21. Photographs showing the adhesion between the crossed hydrogel filaments.

The crossed filaments were prepared by injecting 5 w/v % HyA solution at 2 M Ca²⁺ content and different HCl concentrations of 10-30 mM HCl in 30 mM FeCl₃ solution and reacting for 10 s.

FIGS. 22A-22C. Direct writing of HyA hydrogels in FeCl₃ solution. (A) schematic diagram showing the procedure of direct writing of HyA hydrogels in FeCl₃ solution. HyA solution with H⁺ and Ca²⁺ ions were firstly directly printed into 30 mM FeCl₃ solution, the printed construct was then immersed in 50 mM FeCl₃ solution for 3 min, followed by immersed in DI water for 24 hours. (B) photographs of a 3D construct during the 3D printing. (C) optical micrographs of crosslinked HyA hydrogels that were printed into 30 mM FeCl₃ solution and then immersed in 50 mM FeCl₃ solution followed by immersed in DI water for 24 hours, with both top view (Left) and cross-sectional view (Right) of the 3D-printed hydrogels. The HyA solution for 3D printing was 5 w/v % HyA solution with 30 mM HCl and 2 M Ca²⁺ FIG. 23. Photographs showing the cell culture media (red) is flowing through the hydrogel filament to indicate its tubular structure.

### DETAILED DESCRIPTION

Tunable ratios of mono-, bi- and tridentate coordination in HyA determine the crosslinking density and solid-liquid reversibility of hydrogels

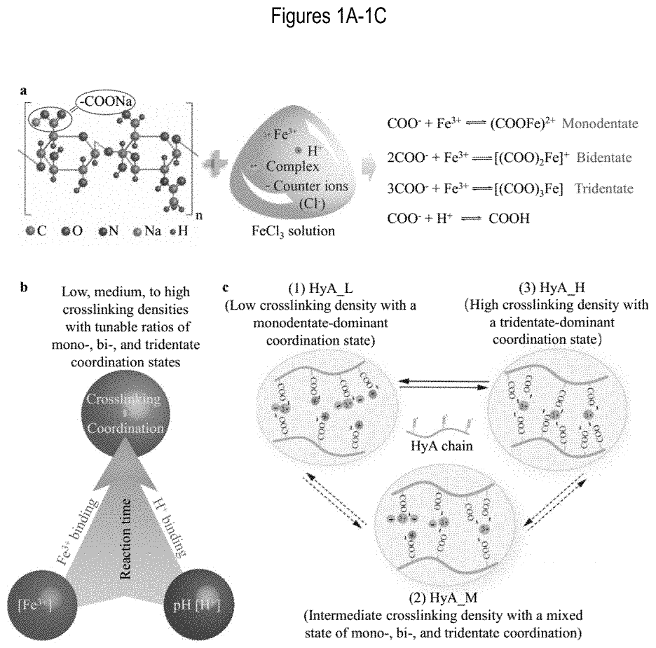

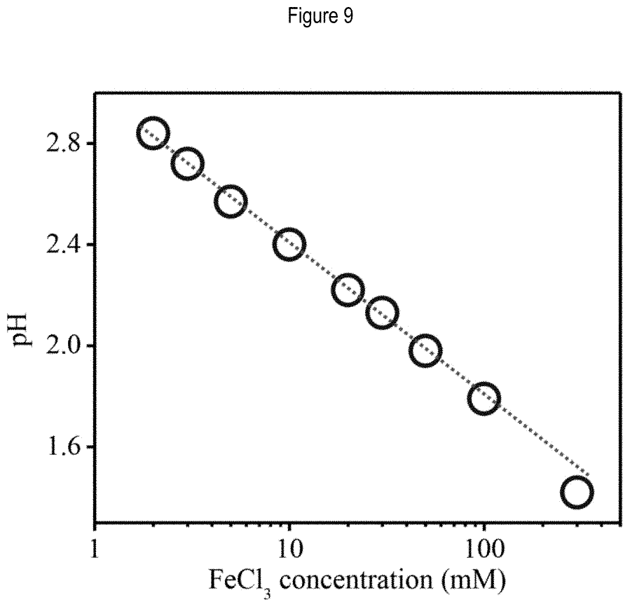

FIG. 1a shows the chemical structure of sodium hyaluronate, which is the salt form of HyA with one carboxyl group per disaccharide unit. When a FeCl₃ solution is added to the sodium hyaluronate, mono-, bi-, and tridentate coordination between Fe³⁺ ions and carboxyl groups of HyA can form as shown in FIG. 1a. The coordination sites of carboxyl groups in HyA can also be occupied by H⁺ ions due to the acidic nature of Fe³⁺ solution. It is well known that ferric iron (Fe³⁺) solution such as iron chloride (FeCl₃) solution is inherently acidic because the electrons between the O—H bond of the water in this solution become polarized until an H⁺ ion from water is liberated (Amira, S., et al., The Journal of Physical Chemistry B 2004, 108 (1), 496-502). In addition to the Fe³⁺ ions and their complex (e.g., [Fe(H₂O)₆]³⁺) (Curtiss, L., et al., Chemical physics 1989, 133 (1), 89-94), the iron salt solution has abundant hydrogen ions (H⁺), thus forming an acidic solution. The innate pH value of FeCl₃ solution (i.e., FeCl₃ solution dissolved in deionized water without pH adjustment) decreases as Fe³⁺ concentration increases; specifically, the innate pH of FeCl₃ solution showed a negative linear relationship with the logarithmic form of Fe³⁺ concentration (FIG. 9). Thermodynamically, both Fe³⁺ and H⁺ (pH) ion concentrations determine the tunable ratios of mono-, bi-, and tridentate coordination states and the resulted low-to-high crosslinking densities of Fe³⁺ crosslinked HyA hydrogels, as shown in FIG. 1b. The equilibrium constant for mono (K₁), bi (K₂), and tridentate (K₃) coordination, and H⁺ (K₄) replacement is defined below respectively:

$$\begin{matrix}{K}_{1} = \frac{[ {Fe}^{3 +} {COO}^{-} ]}{[ {Fe}^{3 +} ] [ {COO}^{- 1} ]} & ( 1 ) \\ {K}_{2} = \frac{[ {{Fe}^{3 +} ( {COO}^{-} )}_{2} ]}{{[ {Fe}^{3 +} ] [ {COO}^{-} ]}^{2}} & ( 2 ) \\ {K}_{3} = \frac{[ {{Fe}^{3 +} ( {COO}^{-} )}_{3} ]}{{[ {Fe}^{3 +} ] [ {COO}^{-} ]}^{3}} & ( 3 ) \\ {K}_{4} = \frac{[ {H}^{+} {COO}^{-} ]}{[ {H}^{+} ] [ {COO}^{-} ]} & ( 4 )\end{matrix}$$

Equation (1a-4a) below are derived from Equation (1-4) to show the ratio of bonded over free carboxylic groups:

$$\begin{matrix}\frac{[ {Fe}^{3 +} {COO}^{- 1} ]}{[ {COO}^{-} ]} = [ {Fe}^{3 +} ] {K}_{1} & ( 1 a ) \\ \frac{[ {{Fe}^{3 +} ( {COO}^{-} )}_{2} ]}{{[ {COO}^{-} ]}^{2}} = [ {Fe}^{3 +} ] {K}_{2} & ( 2 a ) \\ \frac{[ {{Fe}^{3 +} ( {COO}^{-} )}_{3} ]}{{[ {COO}^{-} ]}^{3}} = [ {Fe}^{3 +} ] {K}_{3} & ( 3 a ) \\ \frac{[ {H}^{+} {COO}^{-} ]}{[ {COO}^{-} ]} = [ {H}^{+} ] {K}_{4} & ( 4 a )\end{matrix}$$

Equation (1b-3b) are derived from Equation (1-4) to show the concentration ratio of mono-, bi-, and tri-dentate carboxylate-Fe³⁺ to carboxylate-H⁺ coordination.

$$\begin{matrix}\frac{[ {Fe}^{3 +} {COO}^{-} ]}{[ {H}^{+} {COO}^{-} ]} = \frac{{K}_{1}}{{K}_{4}} \cdot \frac{[ {Fe}^{3 +} ]}{[ {H}^{+} ]} & ( 1 b ) \\ \frac{[ {{Fe}^{3 +} ( {COO}^{-} )}_{2} ]}{[ {H}^{+} {COO}^{-} ]} = \frac{{K}_{2}}{{K}_{4}} \cdot \frac{[ {Fe}^{3 +} ]}{[ {H}^{+} ]} \cdot [ {COO}^{-} ] & ( 2 b ) \\ \frac{[ {{Fe}^{3 +} ( {COO}^{-} )}_{3} ]}{[ {H}^{+} {COO}^{-} ]} = \frac{{K}_{3}}{{K}_{4}} \cdot \frac{[ {Fe}^{3 +} ]}{[ {H}^{+} ]} \cdot {[ {COO}^{-} ]}^{2} & ( 3 b )\end{matrix}$$

Kinetically, however, the intermediate coordination states can also be achieved by controlling the reaction time, as illustrated in FIGS. 1b and 1c. When taking both thermodynamic equilibrium states and kinetically metastable states into consideration, three different coordination states of Fe³⁺ and carboxyl groups in HyA could form. Specifically, as shown in FIG. 1c, HyA hydrogel with low crosslinking density (named as HyA_L) has a monodentate-dominant coordination state, which could form when H⁺ ions replace the Fe³⁺ ions in the coordination site; HyA hydrogel with high crosslinking density (named as HyA_H) has a tridentate-dominant coordination state, which could form when Fe³⁺ ions mainly form tridentate coordination with carboxyl groups; and HyA hydrogel with medium crosslinking density (named as HyA_M) could form as an intermediate state by controlling the reaction time, when carboxyl groups are still partially occupied by H⁺ ions and Fe³⁺ ions also form a mixture of mono-, bi-, or tridentate with carboxyl groups. The crosslinking densities and liquid-solid states of these HyA hydrogels are tunable and reversible because of the tunability and reversibility of carboxylate-Fe³⁺ coordination bonds. The tunability and reversibility of carboxylate-Fe³⁺ coordination were also supported by a study that showed enhanced mechanical properties of poly(acrylamide-co-acrylic acid) (p(AAm-co-AAc)) using carboxylate-Fe³⁺ as secondary crosslinking (Lin, P., et al., Advanced Materials 2015, 27(12), 2054-2059). Specifically, when loading Fe³⁺ ions in a pre-crosslinked p(AAm-co-AAc) hydrogel, Fe³⁺ ions formed a mixture of mono-, bi-, tridentate coordination with carboxyl groups on AAc. After the re-organization of coordinates by immersing the hydrogel in DI water, Fe³⁺ ions formed tridentate coordination with carboxyl groups and achieved enhanced mechanical properties. Moreover, Lee et. al. also reported that the catechol-Fe³⁺ showed monodentate coordination at the pH of 4-5, bidentate coordination at the pH of 7-8, and tridentate coordination at the pH of 10-11 (Lee, J., et al., Macromolecules 2016, 49 (19), 7450-7459).

### The Effects of Fe³⁺ Concentration and pH Value on Tunable Ratios of Mono-, Bi-, and Tridentate Coordination and the Resulted Crosslinking Densities of HyA Hydrogels

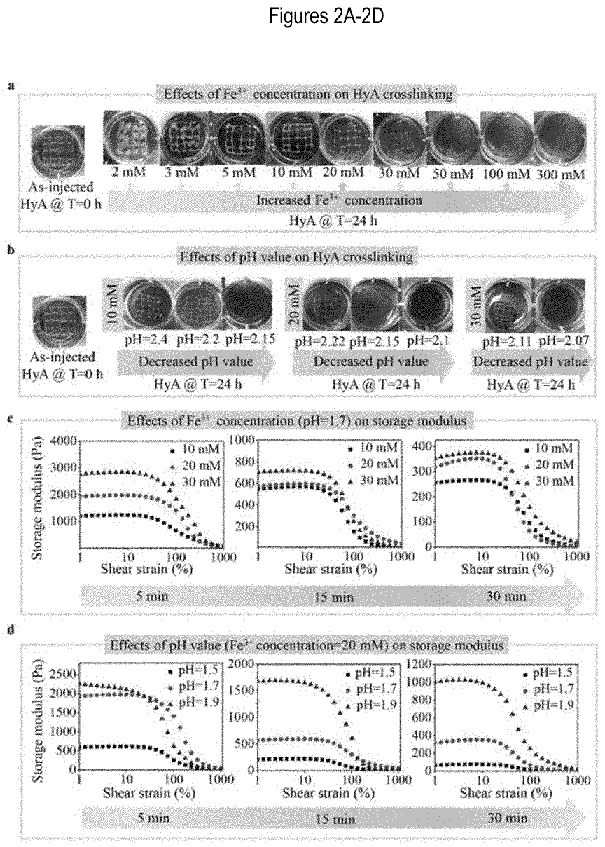

FIG. 2 shows the effects of Fe³⁺ ion concentrations and pH values (H⁺ ion concentrations) on the carboxylate-Fe³⁺ coordination bonding and the resulted tunable crosslinking densities of HyA. As shown in FIG. 2a, a 5 w/v % of HyA solution was injected onto a dish and 2-300 mM FeCl₃ solutions were added. The resulting mixture was allowed to react for 24 hours. The 2-300 mM FeCl₃ solutions were used at their respective innate pH. The HyA exhibited dramatically different structural changes in the FeCl₃ solutions of different concentrations. At the Fe³⁺ concentration of less than 5 mM (2-3 mM), HyA hydrogels swelled and lost the structural integrity. At the Fe³⁺ concentrations of 5-30 mM, HyA hydrogels crosslinked and retained their 3D structure. At the Fe³⁺ concentration of greater than 30 mM (50-300 mM), HyA dissolved in the FeCl₃ solution. To demonstrate the effects of pH on HyA crosslinking, the HyA was crosslinked utilizing a 10-30 mM FeCl₃ solution with pH adjusted to different values using HCl solution. As shown in FIG. 2b, the pH values of 10 mM, 20 mM, and 30 mM FeCl₃ solutions were adjusted to a respective range to achieve the solid or liquid states of HyA after a reaction time of 24 hours, as highlighted using red dashes. When the pH values of 10 mM FeCl₃ solution were adjusted to be ≥2.2 up to the innate pH of 2.4, HyA exhibited as a solid phase. When the pH values were adjusted to be ≤2.15, HyA was in a liquid phase. Similarly, HyA was in a solid phase when the pH values of 20 mM FeCl₃ solution were adjusted to be ≥2.15 up to the innate pH of 2.22, or in a liquid phase when the pH values were adjusted to be ≤2.1. HyA showed as a solid phase when the pH values of 30 mM FeCl₃ solution were adjusted to be >2.11 up to the innate pH of 2.13, or liquid phase when the pH values were adjusted to ≤2.07. The results indicated that in the FeCl₃ solutions with higher Fe³⁺ ion concentration, the lower pH values were required to achieve the liquid phase of HyA.

Furthermore, rheological testing was performed to determine the effects of Fe³⁺ ion concentration, pH value (H⁺ ion concentrations), and reaction time on the storage moduli of HyA hydrogels at the solid or liquid phases. In FIG. 2c, when the pH values of 10 mM, 20 mM, and 30 mM FeCl₃ solutions were adjusted to a constant of 1.7, the storage moduli of HyA hydrogels decreased as the shear strain increased, indicating shear thinning. The storage modulus of the crosslinked HyA hydrogel increased with the increase of Fe³⁺ concentration, which was observed at all time points of 5-15 min. In FIG. 2d, when the Fe³⁺ concentration was fixed at 20 mM, the storage modulus of the crosslinked HyA hydrogel decreased with decreasing pH values. In both FIGS. 2c and 2d, all hydrogel samples exhibited similar shear-thinning property and time-dependent rheological property. The storage moduli of all groups decreased with increasing reaction time from 5 minutes to 15 minutes, revealing the reaction time as a kinetic parameter.

The Fe³⁺ crosslinking of HyA hydrogels mainly involved two dynamic reactions of (1) formation of carboxylate-Fe³⁺ coordination to crosslink the molecular chains of HyA and (2) replacement of Fe³⁺ ions at the sites of coordination bond with H⁺ ions. When H⁺ ions replace the Fe³⁺ ions at the coordination sites, the crosslinking of polymeric chains is partially broken. These two reactions are dynamic and reversible, and eventually reach an equilibrium state. Both the concentrations of Fe³⁺ ion and H⁺ ion (pH value) in the solution determine the equilibrium state and the resulted HyA crosslinking densities, according to the definition of the equilibrium constant (K₁, K₂, K₃, and K₄) described above. FIGS. 1b and 1c illustrate this as well. According to the Equation (1a)-(4a), at the same HyA concentration with innate pH, the Fe³⁺ ion concentration directly determines the ratio of [Fe³⁺COO⁻]/[COO⁻], [Fe³⁺(COO⁻)₂]/[COO⁻]², and [Fe³⁺(COO⁻)₃]/[COO⁻]³. That is, the ratios of [Fe³⁺COO⁻]/[COO⁻], [Fe³⁺(COO⁻)₂]/[COO⁻]², and [Fe³⁺(COO⁻)₃]/[COO⁻]³ increase when the Fe³⁺ ion concentration increase. According to Equation (4a), the H⁺ ion concentration (pH) of HyA solution directly determines the ratio of [H⁺COO⁻]/[COO⁻]. That is, when the pH of HyA solution decreases, the ratio of [H⁺COO⁻]/[COO⁻] increases, because more COO⁻ ligands are bonded with H⁺ ions. The concentration ratios of mono-, bi-, and tri-dentate carboxylate-Fe³⁺ to carboxylate-H⁺ coordination (on the left of Equation 1b-3b) determine the HyA crosslinking density. As shown in Equation (1b-3b), K₁, K₂, K₃, and K₄ are constant when the environmental conditions such as temperature and pressure are the same. According to Equation (1b), the ratio of Fe³⁺ and H⁺ ion concentration, that is, [Fe³⁺]/[H⁺], directly determines the ratio of monodentate [Fe³⁺COO⁻]/[H⁺ COO⁻], when K₁ and K₄ are constants. In the cases of bidentate and tridentate coordination in the Equation (2b) and (3b), both [Fe³⁺]/[H⁺] and the [COO⁻] determine the ratios of bi and tri-dentate coordination states, that is, [Fe³⁺(COO⁻)₂]/[H⁺ COO⁻] and [Fe³⁺(COO⁻)₃]/[H⁺ COO⁻]. The ratios of bi- and tri-dentate coordination states directly define the resulted HyA crosslinking density.

The innate pH of HyA solution decreases when the concentration of HyA increases; and the pH of HyA solution can also be intentionally adjusted to be higher or lower using an acid such as HCl. The relationship between the HyA crosslinking density and the concentrations of Fe³⁺ and H⁺ ions as expressed in Equation (1b-3b) clearly explains the results in FIG. 2a, that is, why HyA was crosslinked to retain its 3D shape in 5-30 mM Fe³⁺ solutions but dissolved in >30 mM (50-300 mM) Fe³⁺ solutions after 24 hours of reaction. Specifically, FIG. 9 shows the innate pH of FeCl₃ solution had a negative linear relationship with the logarithmic form of Fe³⁺ concentration, which suggested that the FeCl₃ solutions with innate pH values have a constant ratio of [Fe³⁺]/[H⁺]. Moreover, at the constant HyA concentration, more free carboxyl groups (COO⁻) became bonded to Fe³⁺ ions when Fe³⁺ concentration increased, as shown in Equation (1a-3a). This means that the concentration of free carboxyl groups ([COO⁻]) decreases as Fe³⁺ concentration increases when HyA concentration does not change. As shown in the right part of Equation (1b-3b), when Fe³⁺ concentration increases and HyA concentration does not change, the ratio of [Fe³⁺]/[H⁺] is a constant, and the concentration of free carboxyl groups ([COO⁻]) decreases. As a result, the ratios of [Fe³⁺(COO⁻)₂]/[H⁺ COO⁻] and [Fe³⁺(COO⁻)₃]/[H⁺ COO⁻] and the resulted HyA crosslinking density decreased, as shown in the left part of Equation (1b-3b). In these cases, when the concentration of FeCl₃ solutions was increased to be higher than 30 mM (50-300 mM) and the HyA concentration did not change, the ratios of [Fe³⁺(COO⁻)₂]/[H⁺ COO⁻] and [Fe³⁺(COO⁻)₃]/[H⁺ COO⁻] and the resulted HyA crosslinking density at the equilibrium state decreased. Thus, the hydrogel transitioned toward the monodentate-dominant coordination state, which resulted in low crosslinking density as seen in the swelling and further dissolution of the HyA in the high concentrations of 50-300 mM FeCl₃ solutions.

Moreover, although HyA was a solid at the equilibrium state in the 10-30 mM FeCl₃ solutions at their innate pH values, as shown in FIG. 2a, the addition of H⁺ ions in these FeCl₃ solutions broke the original equilibriums by replacing Fe³⁺ ions at the coordination sites and drove HyA to a liquid phase at the new equilibrium state as shown in FIG. 2b. To drive HyA to the liquid phase, FeCl₃ solution at the higher Fe³⁺ concentration requires the lower pH values (higher H⁺ ion concentrations) to decrease the ratio of Fe³⁺ to H⁺ ion concentration, thus decreasing the resulted ratios of mono, bi, and tridentate carboxylate-Fe³⁺ to carboxylate-H⁺ coordination, as expressed in the Equation (5b-7b); the results in FIG. 2b confirmed this. Moreover, the formation rate of coordination bonds is determined by the kinetic constant k(T) that is defined below:

k(T)=Ae^{-Eα/RT} (5)

where T is the temperature, A is the constant of proportionality, Eₐ is the activation energy, and R is the gas constant. Upon the addition of FeCl₃ solution to HyA solution, HyA_M with a mix of mono-, bi-, and tri-dentate carboxylate-Fe³⁺ coordination first formed, because the formation of carboxylate-Fe³⁺ coordination has a greater k(T) than carboxylate-H⁺ bonding. As the reaction time increased, Fe³⁺ ions at the coordination sites are gradually replaced by H⁺ ions toward the equilibrium state of coordination bonding. The rheological results confirmed that the reaction time is a kinetic factor. As shown in FIGS. 2c and 2d, intermediate states with varied storage moduli formed at different reaction time of 5-15 minutes before reaching their respective equilibrium states and crosslinking densities, supporting the hypothesis illustrated in FIGS. 1b and 1c.

### Demonstration of the Reversible Solid-Liquid Phase Transition of HyA Hydrogels

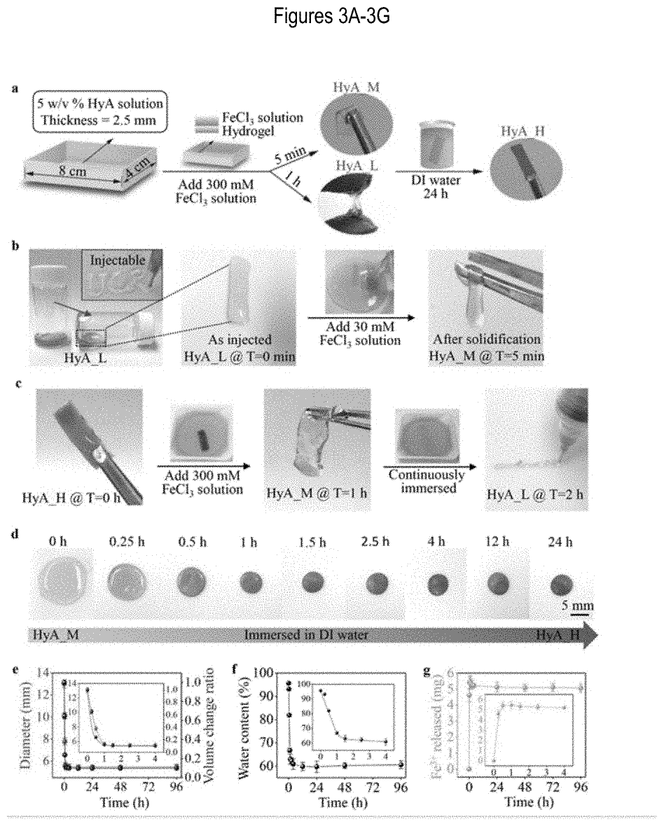

The solid or liquid phase states of the HyA hydrogels are highly tunable and reversible, based on the effects of Fe³⁺ ion and H⁺ ion concentrations on the coordination states and the resulted crosslinking densities in HyA. Reversible phase transitions between HyA_L, HyA_M, and HyA_H were achieved by controlling dynamic carboxylate-Fe³⁺ coordination, that is, adjusting Fe³⁺ and H⁺ (pH) concentrations of the FeCl₃ solutions, as demonstrated in FIG. 3. Specifically, a thin layer of HyA solution with the dimension of 4×8×0.25 cm was cast in a PTFE mold, and 32 mL of FeCl₃ solution was poured into the mold to crosslink the hydrogel. When 300 mM FeCl₃ solution was used in this demonstration, the liquid phase HyA_L formed, based on the conditions established in FIG. 2. As shown in FIG. 3a, formation of HyA_M and HyA_L hydrogels are dependent on the reaction time. After 5 minutes of reaction, the soft HyA_M hydrogel formed and it bent when being held vertically using a. When the reaction time was extended to more than 1 hour, the soft HyA_M transformed to the viscous liquid HyA. Interestingly, both HyA_M and HyA_L transformed to a strong HyA_H hydrogel when being immersed in deionized (DI) water for 24 hours, and the HyA_H did not bend when being held up vertically using a tweezer. HyA_L is a viscous liquid gel with certain fluidity and injectability, as shown in FIGS. 3a and 3b. When a thin 1 mm layer of HyA_L was injected onto a plastic dish in FIG. 3b, a piece of soft and bendable hydrogel formed after 5 minutes of reaction with 20 mL of 30 mM FeCl₃ solution, indicating the phase transition from HyA_L to HyA_M. In FIG. 3c, the stiff HyA_H shows the phase transition to HyA_M and HyA_L. HyA_H swelled in 300 mM FeCl₃ solution and transformed to soft bendable HyA_M after 1 hour of reaction time. After another 1-hour reaction, the transition of solid to liquid phase occurred and a viscous gel HyA_L formed, demonstrating the phase transition from HyA_M to HyA_L. The reversible phase transitions resulted from dynamic carboxylate-Fe³⁺ coordination.

In the phase transition from HyA_M to HyA_H, as shown in FIG. 3d-g, the dimension such as volume or diameter of HyA and the water content in the hydrogel decreased over reaction time, while the Fe³⁺ ion concentration in the HyA hydrogel increased over reaction time. The major changes in HyA dimension, water content and Fe³⁺ ion concentration occurred in the first hour of immersion in DI water and reached plateau after 1 hour. The photographs in FIG. 3d show that the diameter and volume of HyA_M shrank dramatically when immersed in DI water over the period of 0-1 hour and gradually transformed to HyA_H. In FIG. 3e, when the diameter of HyA_M was measured over 96 hours of reaction time, the diameter reduced from 13.02±0.37 mm to 5.63±0.24 mm and the final over initial volume ratio reduced to 0.008 over the first hour, and after 1 hour HyA hydrogel reached an equilibrium state. Similarly, the water content of the hydrogel decreased from 95%±0.14% to 66.78%±0.95% over the first hour and reached a stable state as shown in FIG. 3f. The cumulative release of Fe³⁺ ions from the hydrogel increased to 5.45±0.38 mg over the first hour of immersion in DI water and stabilized afterward, as shown in FIG. 3g, following the reverse trend of the changes in diameter, volume ratio, and water content of the HyA hydrogel.

### The Microstructure, Composition, Thermal, Mechanical, and Rheological Properties of HyA Hydrogels

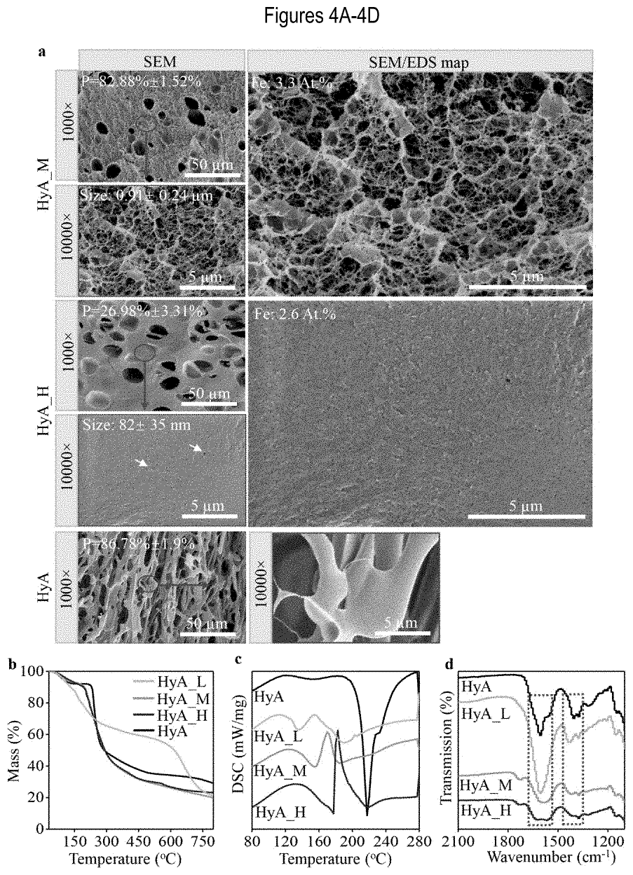

FIG. 4 shows the microstructure, composition, thermal stability, and glass transition temperature of HyA_L, HyA_M, HyA_H and, HyA control. As shown in FIG. 4a, scanning electronic microscopy (SEM) images show dramatically different microstructures of the lyophilized HyA_M, HyA_H, and HyA control and Fe distribution. At the original magnification of 1000×, both HyA_M and HyA_H exhibited large pores that randomly distributed on their cross-sections and formed due to lyophilization process. Interestingly, at the same magnification of 10000×, the SEM image of the polymeric matrix of the lyophilized HyA_M shows highly porous networks with pore size of 0.91±0.24 μm, while the polymeric matrix of the lyophilized HyA_H appears much denser with sporadically scattered pores of 82±35 nm in size. The porosity of lyophilized HyA_M was 82.88% 1.52%, which was significantly higher than the lyophilized HyA_H with a porosity of 26.98% 3.31%. This is possibly because HyA_M had medium crosslinking density, relatively looser hydrogel network and more space to retain higher water content in its microstructure when compared with HyA_H with high crosslinking density. The overlaid SEM/EDS maps showed that Fe element distributed around the pores and located on the polymeric network of lyophilized HyA_M, but uniformly dispersed on the dense polymeric matrix of lyophilized HyA_H. The atomic percentage of Fe element was 3.3% on the cross-section of lyophilized HyA_M, and 2.6% on that of HyA_H. The atomic percentage and distribution of Fe element further confirmed that HyA_M formed the highly porous polymeric networks because of its lower crosslinking density than HyA_H. The SEM images of HyA control showed highly porous microstructure at both low and high magnifications (1000× and 10000×) with the porosity of 86.78%±1.9%. The lyophilized HyA control showed a higher porosity than both HyA_M and HyA_H, possibly because 5 w/v % (4.76 wt. %) of HyA solution had a water content of 95.24% and during lyophilization process large pores with high porosity formed.

Thermogravimetric analysis (TGA) in FIG. 4b shows the mass %-temperature curves of lyophilized HyA_L, HyA_M, HyA_H, and HyA control, and varied slopes were observed at distinct temperature ranges. Specifically, HyA_L first underwent a continuous mass loss of 12% from 50° C. to 150° C., followed by a second slope of 35% mass loss from 150° C. to 600° C., and the third slope of 32% mass loss was from 600° C. to 800° C. After TGA for HyA_L, 21% of sample mass remained. The mass %-temperature curves of HyA_M and HyA_H were similar with significant overlap, and both samples showed a slope of 12% mass loss from 50° C. to 200° C., followed by a second slope of 53% mass loss from 200° C. to 400° C., and then a reduced slope of 15% mass loss for HyA_M and 12% for HyA_H at 400° C. to 800° C. The residual sample mass after TGA was 20% for HyA_M and 23% for HyA_H. HyA control showed a mass loss of 8% from 50° C. to 150° C., and the mass remained stable at 50° C. to 230° C. Subsequently, a slope of 41% mass loss at 230° C. to 300° C. and a reduced slope of 12% mass loss at 300° C. to 800° C. were observed for HyA control. The residual mass of HyA control after TGA testing was 29%.

It has been reported that polysaccharide degradation in thermogravimetric analysis (TGA) proceeds in four steps, including (1) evaporation of free moisture, (2) dehydration of bonded water, (3) decomposition accompanied by the rupture of C—O and C—C bonds in the ring units resulting in the evolution of CO, CO₂ and H₂O, and (4) formation of polynuclear aromatic and graphitic carbon structures. Because all of the samples have been lyophilized to remove free moisture before testing, the weight loss at the first slope was mainly attributed to the dehydration of bonded water for HyA_M, HyA_H and HyA control. However, for HyA_L, the hydrogel underwent a swelling process and absorbed a large amount of water in its network during the phase transition from HyA_M to HyA_L, and the Fe³⁺ and Cl⁻ ions dissolved in the absorbed water remained in the lyophilized HyA_L. Therefore, the lyophilized HyA_L contained a much higher amount of residual FeCl₃ particles than the lyophilized HyA_M and HyA_H. The residual FeCl₃ can possibly react with the released water from both FeCl₃.6H₂O and HyA polymeric chain at 100° C. to 200° C. to form Fe(OH)₃ and HCl gas. The mass loss in the first slope of HyA_L was therefore possibly due to the dehydration of bonded water and the evaporation of HCl gas. At the temperature higher than 200° C. for HyA_M and HyA_H and higher than 230° C. for HyA, the mass loss was mainly caused by the decomposition process of the HyA polymeric phase. For HyA_L, the mass loss after 150° C. mainly resulted from the decomposition of HyA chains and Fe(OH)₃, and a small amount of bonded water and HCl gas could be released at 150° C.-200° C. It has been reported that Fe(OH)₃-polyacrylamide hybrid polymer had a dramatic weight loss from 575° C. to 675° C. because the ionic bond formed between Fe(OH)₃ and polyacrylamide chains increased the thermal stability of the hybrid, which could possibly explain the second slope of rapid mass loss of HyA_L after 600° C. The remaining sample mass is mainly composed of polynuclear aromatic and graphitic carbon materials after decomposition.

Moreover, the residual materials of HyA_L, HyA_M, and HyA_H groups may also contain Fe₂O₃, and HyA residual mass may contain Na₂CO₃. As shown in the differential scanning calorimeter (DSC) results in FIG. 4c, the glass transition temperature (Tg) was 125° C. for HyA_L, 142° C. for HyA_M, 150° C. for HyA_H, and 140° C. for HyA. Generally, hydrogels with higher crosslinking density will exhibit a higher Tg. The Tg of HyA hydrogels increased from HyA_L, to HyA_M and HyA_H when the crosslinking densities of these three types of HyA hydrogels increased. Tg of HyA_L, however, was much lower than HyA group. which may be attributed to the relatively high content of residual FeCl₃ salt particles. It has been reported that the Tg of polymer nanocomposites can increase or decrease when the concentration of nanoparticles increases, depending on the specific physical or chemical interactions between the nanoparticles and polymer matrix. For example, the alumina nanoparticles in PMMA have been found to reduce Tg of PMMA nanocomposite, and alumina is known to have limited interactions with PMMA. Similarly, the high content of FeCl₃ particles in HyA_L may lead to the reduction of Tg, because the residual FeCl₃ salt particles in lyophilized HyA_L are expected to have limited interactions with HyA matrix.

FIG. 4d shows the FTIR-ATR spectra of HyA_L, HyA_M, HyA_H, and HyA control. As highlighted in the red rectangles, peaks appeared in the zone between 1200 cm⁻¹ and 1800 cm⁻¹ are assigned to the asymmetric and symmetric stretching vibrations of carboxyl groups. For the frequency shift, where Δv=v_{asym}−v_{sym}, both HyA_L and HyA have a Av of 230; the value varied in other samples where Δv=205 for HyA_M and Δv=190 for HyA_H. The similarity of the spectra between HyA_L and HyA in the highlighted zoon may be attributed to the fact that both samples have monodentate-dominant coordination states. The differences among the peaks of HyA_L, HyA_M, and HyA_H suggested the change of carboxylate-Fe³⁺ coordination states, further supporting the transition of dynamic coordination bonds illustrated in FIG. 1c.

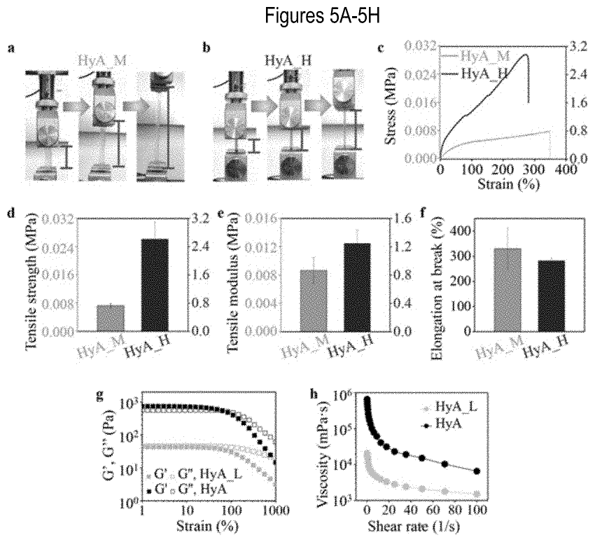

The coordination state determines the crosslinking density and microstructure of the hydrogels, which plays a significant role in their mechanical properties. FIGS. 5a and 5b show the photographs of HyA_M and HyA_H during tensile testing; they were both highly stretchable. The representative stress-strain curves of HyA_M and HyA_H in FIG. 5c demonstrated their huge difference in mechanical properties. Specifically, in FIG. 5d, the tensile strength of HyA_H reached 2.62±0.49 MPa, which was almost three orders of magnitude higher than HyA_M with a tensile strength of 7.3±0.65 kPa.

In FIG. 5e, similar to tensile strength, the tensile modulus of HyA_H was 1.25+0.19 MPa, while HyA_M was 8.66±1.81 kPa. In terms of elongation in FIG. 5f, HyA_M had an elongation at break of 330±81.85%, while that of HyA_H was 282.00±11.53%. The tri-dentate coordination state and the resulted high crosslinking density in HyA_H led to the significantly higher tensile strength and elastic modulus of HyA_H than HyA_M. The high elongation at break for both HyA_M and HyA_H was attributed to the dynamic coordination that provided the hydrogel with reversible bonding to dissipate the mechanical energy and increase deformability.

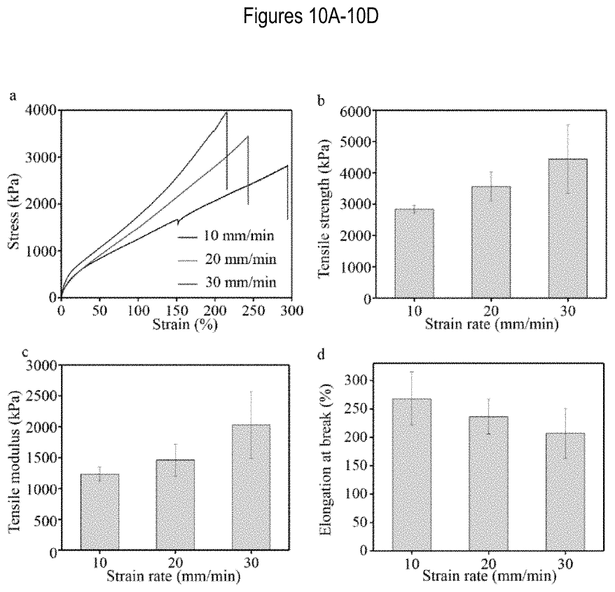

This mechanism of reversible bonding has also been applied in acrylic polymers to enhance the mechanical properties. For example, carboxylate-Fe³⁺ coordination was utilized as secondary crosslinking to further strengthen the p(AAm-co-AAc) hydrogel which reached a tensile strength of 5.9 MPa. Moreover, different strain rates utilized in the tensile testing dramatically affect the performance of the hydrogels. FIG. 10 shows the tensile testing results of HyA_H at different strain rates of 10 mm/min, 20 mm/min, and 30 mm/min. Specifically, as the strain rate increased, tensile strength and tensile modulus of the HyA_H hydrogels increased but the elongation at break decreased, as shown in FIG. 10a-10d This is mainly because the reversible coordination bonding in HyA hydrogels is capable of dissipating the mechanical energy generated during the tensile stretching, and the varied strain rates used for tensile testing affect the time available for energy dissipation. For example, at a lower strain rate, more time would be available for reversing the bonding and dissipating the mechanical energy; and thus, the hydrogels should have lower tensile strength and modulus and higher elongation at break in the tensile testing.

The rheological properties of HyA_L and HyA in FIGS. 5g and h showed that both HyA_L and HyA experienced shear-thinning. In FIG. 5g, storage modulus and loss modulus of HyA_L and HyA decreased when the shear strain increased. In FIG. 5h, the viscosities of HyA_L ranged from 20514 mPa·s to 1499 mPa·s at the shear rate of 0.1 l/s to 100 l/s, and HyA showed the viscosities from 655000 mPa·s to 6498 mPa·s at the shear rate of 0.1 l/s to 100 l/s.

HyA_L with a low crosslinking density absorbed more water in its network during solid-liquid phase transition, and thus, had lower HyA concentration than the HyA control group at 5 w/v % (4.76 wt. %). As a result, HyA_L had a lower dynamic modulus and viscosity than HyA control.

### Three-Dimensional Printing of HyA Hydroge

Effects of Fe³⁺ and H⁺ ion concentrations and reaction time on 3DP or HyA

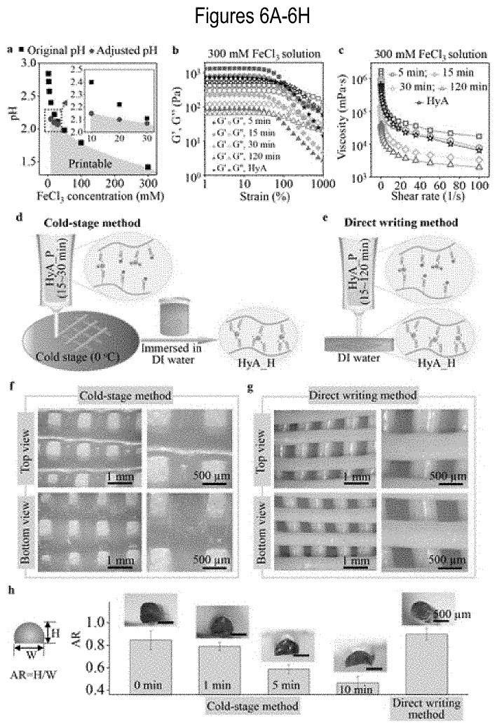

The feasibility of 3DP with of HyA was demonstrated via dynamic coordination of carboxylate-Fe³⁺ ion, inspired from the tunable crosslinking density and reversible phase transition capability of HyA. Traditionally, unmodified HyA solution is not considered to be 3D printable because the limited structural stability of the printed constructs. However, HyA hydrogels had tunable crosslinking densities and reversible phase transition capability, enabling 3DP under certain conditions. FIG. 6a shows the conditions of Fe³ concentration and pH value at which the solid-to-liquid phase transition can be achieved. Coordination states and crosslinking densities for HyA hydrogels that are in between HyA_L and HyA_M, were identified. In this range, HyA gels have good injectability under certain shear strain due to the shear thinning; the resulting 3DP structures have good structural stability. The 3D printable HyA gel with the suitable range of coordination states and crosslinking densities was named HyA_P. Theoretically, FeCl₃ solution at the concentrations and pH values in the green region in FIG. 6a can produce HyA_P with the coordination state between HyA_L and HyA_M. Moreover, HyA_P can be obtained by (1) reversing the crosslinking from HyA_H or HyA_M to HyA_P via the reversible phase transition, or (2) directly crosslinking the HyA to HyA_P under specific conditions.

Specifically, HyA_M or HyA_H was immersed in FeCl₃ solutions at the concentrations and pH values in the green region of FIG. 6a, for a specific suitable reaction time, to reverse the crosslinking to achieve HyA_P. The suitable reaction time is dependent on the specific concentration and pH of FeCl₃ solutions. To demonstrate, a 5 w/v % HyA solution was injected into a 300 mM FeCl₃ solution with an innate pH of 1.3 to form HyA_M immediately, and then the reaction time was extended to 5-120 minutes to achieve HyA_P. FIGS. 6b and 6c shows the various rheological properties of partially crosslinked HyA_P that was produced with the extended reaction time of 5 minutes (pink), 15 minutes (purple), 30 minutes (blue), and 120 minutes (dark yellow), respectively, and HyA control (dark). Storage modulus (G′), loss modulus (G″), and viscosity of the various HyA_P all decreased when the reaction time increased. For all samples, G′ and G″ decreased when the shear strain increased as shown in FIG. 6b. Viscosity also decreased when the shear strain rate increased as shown in FIG. 6c. The partially crosslinked HyA_P and HyA control all exhibited a solid state at low shear strain range (G′>G″). The “gel point” (G′=G″) of the partially crosslinked HyA_gels at different reaction time was 156% at 5 minutes, 118% at 15 minutes, 100% at 30 minutes, and 49% at 120 minutes. In contrast, the “gel point value” for the 5 w/v % HyA control was 73%. The results further confirmed that at the specific Fe³⁺ concentration and pH value, the rheological properties (G′, G″, and viscosity) of partially crosslinked HyA_P are controllable and tunable, by adjusting reaction time of HyA_M or HyA_H in FeCl₃ solution.

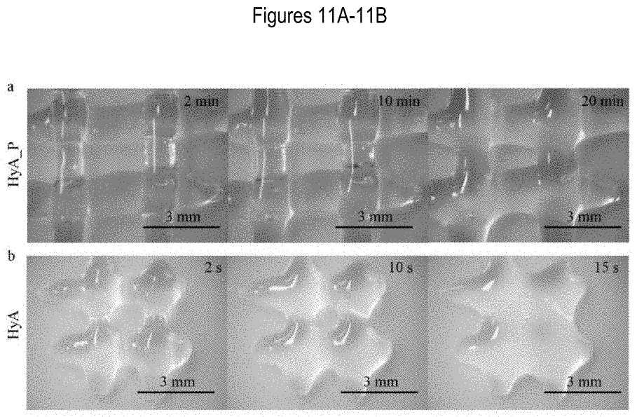

A balance between the structural stability and injectability should be achieved for 3DP of hydrogels. Hydrogels that require a higher shear strain to reach the gel point in FIG. 6b should have a higher structural stability. For example, the HyA_P produced at 15-minutes reaction time was found to have better structural stability than HyA (FIG. 11). Specifically, when printed or injected onto the surface of a petri dish, HyA_P produced using 15-min reaction time still maintained its structure after 20 min (FIG. 11a) while the HyA control lost its original architecture after 15 s (FIG. 11b). However, the hydrogels with high viscosity may require higher force or pressure to inject or extrude for 3DP, which poses additional requirements for 3DP instruments.

As demonstrated in FIGS. 6d and 6e, two strategies were used for 3DP of HyA hydrogels, a cold-stage method and a direct writing method. In FIG. 6d., HyA_P was printed on a cold stage at 0° C. to enhance the stability of the printed, and then the printed constructs were immersed in DI water to achieve stable HyA_H. In FIG. 6e, HyA_P was directly printed in DI water, utilizing the phase transition from HyA_P to HyA_H in water and the supporting benefit of water to enhance the structural stability of the printed constructs. The requirements on the rheological properties of the HyA_P were different in these two 3DP methods. The cold-stage method requires HyA_P to have high structural stability and good injectability, and thus, a reaction time in 300 mM FeCl₃ solution should be 15 minutes to 30 minutes to obtain HyA_P. In contrast, the direct writing method reduces the requirements for structural stability and accepts the HyA_P with a wider range of structural stability, because the phase transition from HyA_P to HyA_H in water provide additional structural stability during 3DP. Thus, the suitable reaction time in 300 mM FeCl₃ solution should be 15 minutes to 120 minutes to obtain HyA_P for direct writing.

Morphology and Structural Stability of 3D Printed HyA Hydrogels Via Cold-Stage and Direct Writing Methods

The optical micrographs in FIG. 6f show the top and bottom view of the printed HyA hydrogels via cold-stage method, and the printed structure appeared similar to what was designed. The images presented that the printed filament on the top layer maintained the original cylindrical shape well while the filament on the bottom layer exhibited flat. The deformation of filaments on the bottom was attributed to the spreading of the gel on the substrate during the printing process. The magnified image shows that the intersection of two filaments at neighboring layers deformed due to the surface tension. The optical micrographs in FIG. 6g exhibits the hydrogels printed using direct writing. The filaments of hydrogels presented similar morphology at both top and bottom view, and all maintained original cylindrical shape after printing. Moreover, the intersection of two layers from neighboring layers also maintained ideal cylindrical structure, further indicating the significant structural stability enhancement effects of the phase transition from HyA_P to HyA_H and water on the 3DP. FIG. 6h shows a comparison of the cross-sections for the hydrogel filaments printed via the cold-stage method and the hydrogel filaments printed via the direct writing method.

For filaments prepared by cold-stage method, gels were kept for 0 minutes, 1 minute, 5 minutes, and 10 minutes, respectively, at room temperature and pressure, after printing, and were then immersed in DI water for 24 hours. The representative micrographs show that the filament printed on the cold stage gradually deformed due to the gravity. The quantitative height/width ratio of the cross-section reduced with time for the samples that were printed using cold-stage method and were kept in room conditions for different time, specifically, 0.85+0.08 for 0 minutes, 0.79±0.04 for 1 minute, 0.59±0.04 for 5 minutes, and 0.47±0.05 for 10 minutes. In contrast, the cross-section of filament printed by direct writing method showed near-circular shape with a height/width ratio of 0.9±0.05. Thus, the hydrogels printed in DI water retained their structure better and more closely to the designed structure.

It is also possible to directly crosslink HyA to form partially crosslinked HyA_P, that is, skipping the reverse phase transition from HyA_M or HyA_H. HyA solution with reduced pH will make the H⁺ ions occupy the coordination sites first. After the addition of Fe³⁺ ions, the Fe³⁺ ions will need to replace the H⁺ ions at the coordination sites and eventually achieve the equilibrium. In this scenario, only the thermodynamic factors need to be considered, the exact pH of HyA solution and the concentration of FeCl₃ can be determined based on the results in FIG. 2 and the relationship between crosslinking density and the concentrations of Fe³⁺ and H⁺ ion, as shown in Equation (1b-3b). For example, HyA solution with reduced pH can be injected directly to 5-30 mM FeCl₃ solution. The final pH value of the hydrogel should be lower than the innate pH of the 5-30 mM FeCl₃ solution but higher than innate pH of 50 mM FeCl₃ solution. HyA solution can also be directly printed with reduced pH into 5-30 mM concentration range of FeCl₃ solution. It is difficult to directly print HyA solution with innate pH values into FeCl₃ solution, because the instant crosslinking of HyA hydrogel prevents bonding of printed filaments. However, when injecting HyA solution with reduce pH into FeCl₃ solution, the large amount of H⁺ ions initially occupy the coordination sites and slow down the hydrogel crosslinking; this allows time for the printed filaments to bond to one another in the layer-by-layer structure.

### Cytocompatibility of HyA Hydrogels in BMSC Culture

Cell Morphology and Adhesion after Culture with HyA Hydrogels and Controls

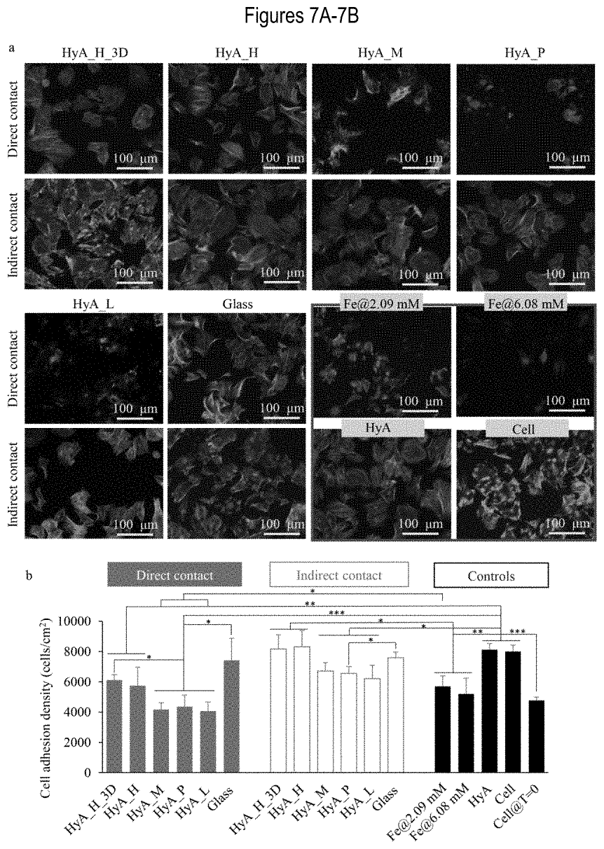

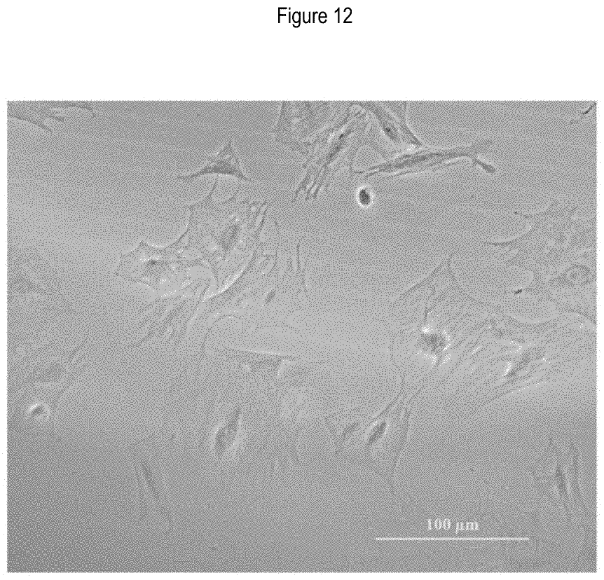

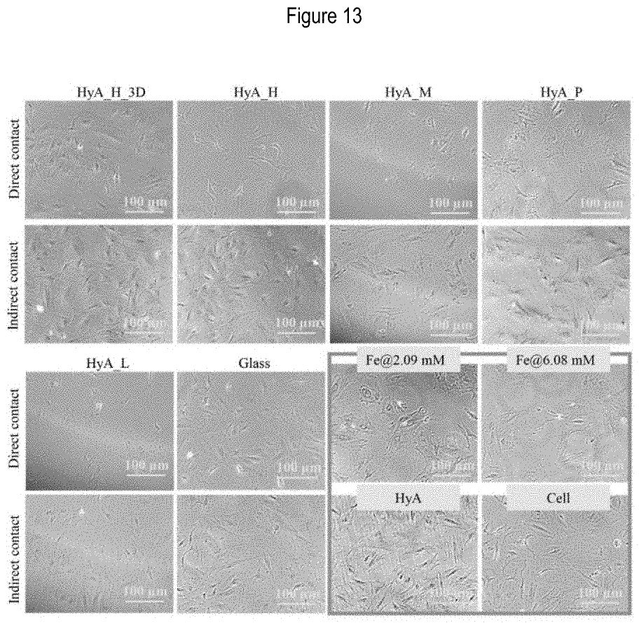

In direct exposure culture, hydrogels were loaded on top of the adhered/established cells. The optical image in FIG. 12 shows the morphology of adhered cells after 24-hour culture before adding the samples. FIG. 7a shows the representative fluorescence images of BMSCs adhered on the well plate and directly in contact with the samples (Direct contact) and BMSCs adhered on the well-plate but surrounding the respective materials (Indirect contact) after a 24-hour direct exposure culture. For all hydrogel groups, the number of cells under indirect contact conditions was larger than cells in direct contact conditions. The fluorescence images also showed that the groups of HyA_H_3D and HyA_H had more cells than the groups of HyA_M, HyA_P, and HyA_L, under both direct and indirect contact conditions. Moreover, for cells under direct contact conditions in the groups of HyA_M, HyA_P, and HyA_L and cells in Fe@2.09 mM and Fe@6.08 mM groups, some cells only show the fluorescence of nuclear but the fluorescence of cell membrane (F-actin) is missing. Optical images in FIG. 13 shows the morphology of BMSCs under direct and indirect contact conditions in HyA hydrogel groups and the control groups of Fe@2.09 mM, Fe@6.08 mM, Glass, HyA, and Cell. BMSCs all adhered and spread on the well-plate despite varied cell numbers between different groups. The missing of the cell membrane fluorescence in FIG. 7a thus maybe because the Fe(OH)₃ formed in the media disturbed the staining of the Alexa Flour 488-phalloidin agent.

FIG. 7b shows the quantitative BMSC adhesion density of different hydrogel samples and control groups. The results show that cell adhesion densities in direct contact conditions for all hydrogel groups of HyA_H_3D, HyA_H, HyA_M, HyA_P, and HyA_L and cell adhesion densities under indirect contact conditions in the groups of HyA_M, HyA_P, and HyA_L were significantly lower than HyA control of 8123±429 cells/cm² and Cell control of 8002±477 cells/cm².

Moreover, for all hydrogel groups of HyA_H_3D, HyA_H, HyA_M, HyA_P, and HyA_L, cell adhesion densities in direct contact conditions were significantly lower than cell adhesion densities under indirect contact conditions. Specifically, in direct contact conditions, the cell adhesion density ratios of hydrogel groups to Cell control were 76.30% for HyA_H_3D, 71.73% for HyA_H, 52.11% for HyA_M, 54.44% for HyA_P, and 50.83% for HyA_L. However, under indirect contact conditions, the ratios were 102.11% for HyA_H_3D, 103.91% for HyA_H, 83.8% for HyA_M, 81.95% for HyA_P, and 77.63% for HyA_L. On average, groups of HyA_H_3D and HyA_H had higher cell adhesion densities under both direct and indirect conditions than groups of HyA_M, HyA_P, and HyA_L. Moreover, cell adhesion densities of HyA and Cell control groups were similar, but both were statistically higher than Fe³⁺ ion control groups of Fe@2.09 mM and Fe@6.08 mM. Thus, the lower cell adhesion densities in hydrogel samples than Cell control can be attributed to the Fe³⁺ and H⁺ ions released from these samples.

HyA Degradation and Associated Changes in Culture Media

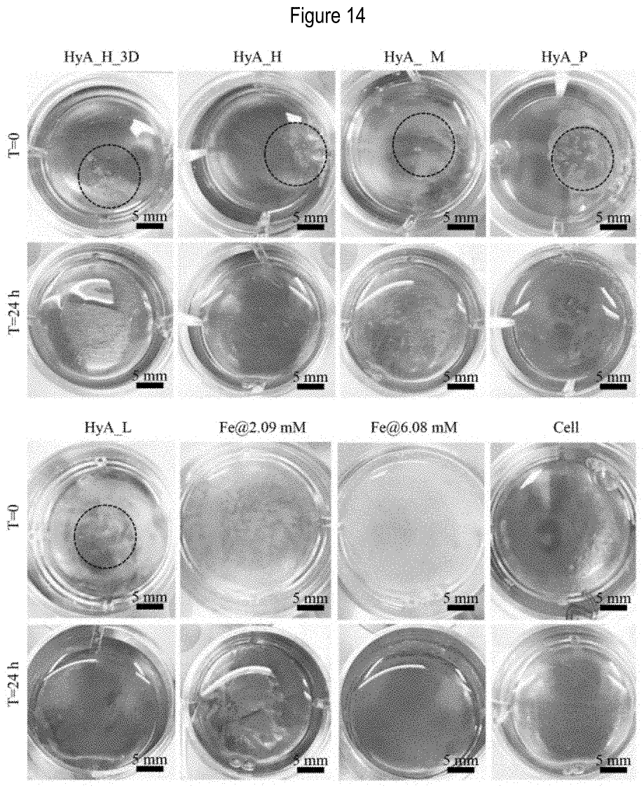

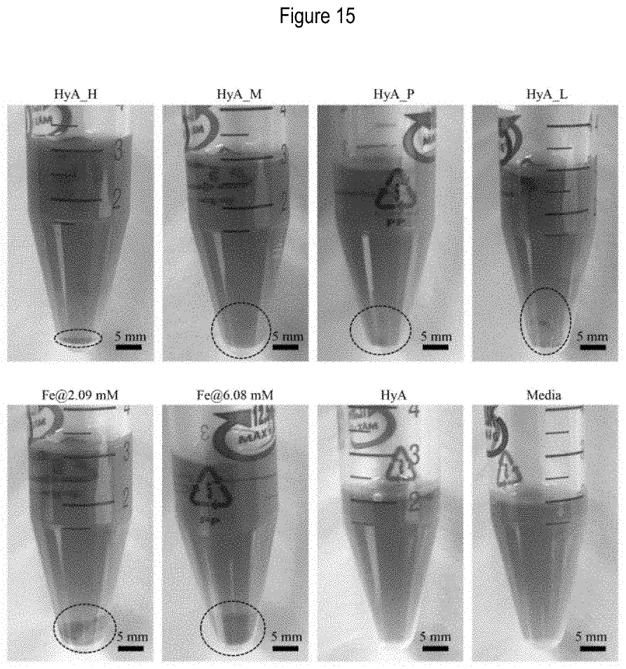

After 24 hours in cell culture, solid HyA hydrogels with tridentate or bidentate coordination (HyA_H_3D, HyA_H, and HyA_M) lost their integrity in the media (FIG. 14), and some fragments of the hydrogel were found in the media (as highlighted in dashed circles in FIG. 15), which indicated partial solid-liquid phase transition. In the cell culture media, there are multiple types of metal ions, such as Ca²⁺, Mg²⁺, Na⁺, and K⁺ ions. Similar to the H ions, these cations can also be bonded to the carboxyl groups of the HyA molecule and replace carboxylate-Fe³ tridentate or bidentate coordination, thus resulting in a decrease of crosslinking density. In general, the reversibility of a hydrogel crosslinked via metal-ligand coordination is dependent on the equilibrium constant (K_{eq}) of the coordination bonds. If the K_{eq} value is excessively large (e.g., >10⁴⁰), the hydrogels are considered to be too stable to be characterized by a “break-after-recovery” behavior.^{32, 52} In such conditions, the hydrogel networks may be considered irreversible. Although the reversible solid-liquid phase transitions of HyA hydrogels were demonstrated under certain conditions (FIG. 3), HyA hydrogels crosslinked via tridentate or bidentate coordination may not be fully reversible in certain conditions such as in the cell culture conditions The results in FIG. 3, S6, and S7 confirmed that the solid-liquid phase transition and reversibility can be achieved at certain conditions, when carboxylate-Fe³⁺ coordination states were fully or partially reversed.

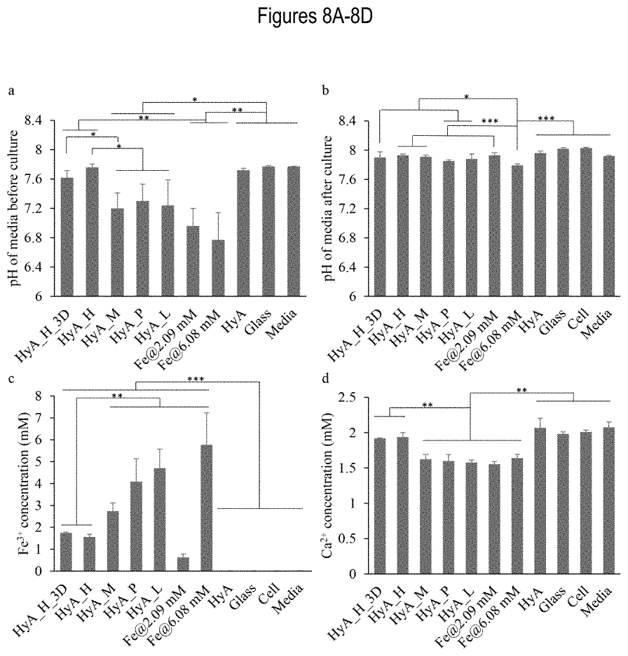

The changes in the pH values and ion concentrations of the media can significantly affect cell behavior. As shown in FIGS. 8a and 8b, the average pH values of HyA_M, HyA_P, HyA_L, Fe@2.09 mM, and Fe@6.08 mM were lower than all other groups before culture, while the pH values of all the groups except Fe@6.08 mM were in a small range of 7.85-7.93 after 24-hour culture. The neutralized pH in media after cell culture is benefited from the bicarbonate buffering system in the cell culture media. Similarly, Cheyann Lee Wetteland et al⁵³ also reported that when adding different concentrations of MgO and Mg(OH)₂ nanoparticles in cDMEM, DMEM, SBF, HEPES, NaCl solution, MgCl₂ solution, and DI water, the changes in the pH values in cDMEM, DMEM, and SBF before and after a 24-hour immersion were significantly smaller than HEPES, NaCl solution, MgCl₂ solution, and DI water. As expected, FIG. 8c shows that the Fe³⁺ concentrations of media in HyA_H_3D and HyA_H groups were significantly lower than HyA_M, HyA_P, and HyA_L but higher than the control groups of HyA, Glass, Cell, and Media. FIG. 8d shows the Ca²⁺ concentration in the post-culture media. The groups of HyA_M, HyA_P, HyA_L, Fe@2.09 mM, and Fe@6.08 mM showed significantly lower Ca²⁺ concentrations than any other groups. It has been reported that Al(OH)₃ and Fe(OH)₃ can uptake (absorb) cations such as Ca²⁺ and Cd²⁺.⁵⁴ Because the groups of HyA_M, HyA_P, HyA_L, Fe@2.09 mM, and Fe@6.08 mM have higher theoretical Fe³⁺ ions and the resulted Fe(OH)₃ content than other groups, the lower Ca²⁺ concentrations in the media of these groups may be attributed to the absorption effects of Fe(OH)₃.

Discussion and Future Directions of HyA Hydrogels for Medical Applications

The lower cell adhesion densities in hydrogel samples than Cell control can be attributed to the Fe³⁺ and H⁺ ions released from these samples. During the culture, Fe³⁺ ions released from the hydrogel samples were diluted in the media, and the acidity of the hydrogel samples was greatly neutralized by the bicarbonate buffering system in the media. However, before the hydrogel samples lost their integrity and dispersed in the media, the high Fe³⁺ concentration and low pH in the region closely surrounding the samples probably affected the cells in a closer distance more than the cells in a further distance due to the dynamic gradient of Fe³⁺ and H⁺ concentrations. This explained why cell adhesion densities in direct contact conditions were significantly lower than cell adhesion densities under indirect contact conditions for all hydrogel groups, as shown in FIG. 7b.

Among the HyA hydrogels, HyA_H_3D and HyA_H showed higher average adhesion densities under direct and indirect contact conditions than other hydrogel groups due to the lower Fe³⁺ containing and acidity, showing potentials for medical applications such as tissue repair. Moreover, for the HyA hydrogels utilized in the cell study, we did not use the optimal Fe³⁺ and H⁺ concentration based on the principles established in the equations. As we mentioned above, both the Fe³⁺ and H⁺ ion concentrations can be reduced to minimize their toxicity concern when crosslinking the hydrogels, which may significantly improve the cytocompatibility of the hydrogels for medical applications in the future.

The following Examples are non-limiting.

### EXAMPLES

### Determine the Effects of Fe³⁺ Concentration and pH Value on Crosslinking and Reversible Phase Transition of HyA Hydrogels

Determine the Effects of Fe³⁺ Concentration on Crosslinking and Reversible Phase Transition of HyA Hydrogels

Sodium hyaluronate (abbreviated as HyA; Bulk Supplements, Henderson, Nev.) solution at the concentration of 5 w/v % was injected into a 6-well plate to create a network pattern with 16 of 8×8 mm squares in a 32×32 mm square. A syringe with a needle size of 0.35 mm was utilized for injection. After creating the HyA network pattern, 10 mL of Fe³⁺ solution (FeCl₃, #169430010, Sigma-Aldrich, St. Louis, Mo.) at the concentrations of 2 mM, 3 mM, 5 mM, 10 mM, 20 mM, 30 mM, 50 mM, 100 mM, and 300 mM was added in different wells to crosslink the HyA, respectively. After 24-hour immersion, the crosslinking states of respective HyA gels were photographed.

Determine the Effects of pH Value on Crosslinking and Reversible Phase Transition of HyA Hydrogels

FeCl₃ solution at the concentrations of 10 mM, 20 mM, and 30 mM was prepared. The FeCl₃ solution at 10 mM, 20 mM, and 30 mM was adjusted to the respective pH range of 2.1-2.4, 2.05-2.3, and 2-2.2, respectively using hydrochloric acid (HCl). HyA network patterns were created in a 6-well plate as described above, 10 mL of FeCl₃ solution at 10 mM, 20 mM, and 30 mM was then added to the respective wells. After 24-hour immersion, the crosslinking states of HyA gels were photographed.

Determine the Effects of Fe³⁺ Concentration and pH Value on Storage Modulus of Crosslinked HyA Gels

To investigate the effects of Fe³⁺ concentration on storage modulus of crosslinked HyA gels, we prepared FeCl₃ solution at the concentrations of 10 mM, 20 mM, and 30 mM, and the pH value of these solutions was intentionally adjusted to 1.7 using HCl. HyA solution at the concentration of 5 w/v % was injected into the respective FeCl₃ solutions of 10, 20, and 30 mM using a syringe with a needle of 0.35 mm in diameter. At the respective time points of 5 min, 15 min, and 30 min, the crosslinked HyA gels were collected using a spoon and tested for storage moduli using a rheometer (MCR 92 with PP25 measuring system, Anton Paar), respectively. For rheological testing, around 1 mL of each collected gel sample was added onto the stage of the rheometer, and then the testing spindle (PP25, Anton Paar) was moved down and the extra gel sample was removed. The gap between the bottom plane of the spindle and stage was set as 1 mm. The strain angular frequency was set as 10 l/s for all the samples, and the storage modulus of the sample at the shear strain of 1%˜1000% (or shear strain of 0.01-10) was recorded at 25° C.

To investigate the effects of pH value on storage modulus of crosslinked HyA gels, we prepared FeCl₃ solution at the concentration of 20 mM and intentionally adjusted its pH value to 1.5, 1.7, and 1.9 using HCl, respectively. HyA solution at the concentration of 5 w/v % was injected into the 20 mM FeCl₃ solutions with respective pH of 1.5, 1.7, and 1.9 following the same method described above. The crosslinked HyA gel was collected and tested for storage modulus using the same rheometer setup.

### Demonstrate the Tunable Crosslinking and Reversible Phase Transition of HyA Hydrogels

Based on different crosslinking degree (or coordination states), the crosslinked HyA gels are classified as HyA hydrogel with low (HyA_L), medium (HyA_M), and high (HyA_H) crosslinking degree.

Demonstrate the Phase Transition from HyA_M to HyA_L:

For this, 8 mL sodium hyaluronate (abbreviated as HyA; Bulk Supplements, Henderson, Nev.) solution at the concentration of 5 w/v % was injected into a PTFE mold (Bottom size: 4×8 cm) using a syringe with a needle of 0.84 mm in diameter. It took 10 min for the viscous HyA solution to form a uniform HyA layer with a thickness of 0.25 mm on the bottom of the mold. After that, 32 mL of FeCl₃ (#169430010, Sigma-Aldrich, St. Louis, Mo.) solution at the concentration of 300 mM was added into the mold to crosslink the HyA. The crosslinking degree of HyA can be controlled by the reaction time with FeCl₃ solution. When the reaction time was 5 min, HyA_M formed. When the reaction time was 1 hour, HyA_L formed.

Demonstrate the phase transition from HyA_M to HyA_H and HyA_L to HyA_H: To obtain HyA_H, the as-prepared HyA_M or HyA_L was immersed in DI water in a beaker for 24 hours at the room temperature and pressure. The DI water was refreshed every 12 hours.

Demonstrate the phase transition from HyA_L to HyA_M: HyA_L is injectable and can be used to directly write letters or patterns. To obtain HyA_M, HyA_L was first injected onto a plastic weighing dish using a syringe with a needle of 0.84 mm in diameter to form a 1 mm-thin layer of the hydrogel, and then 20 mL FeCl₃ solution at the concentration of 30 mM was added on top of the HyA_L to completely cover the HyA_L layer. HyA_M formed after HyA_L reacted with 30 mM FeCl₃ solution for 5 mins at room temperature and pressure.

Demonstrate Phase Transition from HyA_H to HyA_M and HyA_M to HyA_L:

When HyA_H was immersed in 300 mM FeCl₃ solution in a plastic weighing dish for 1 hour, HyA_H transformed to HyA_M. When HyA_H was immersed in 300 mM Fe C13 solution in a plastic weighing dish for 2 hours, HyA_H transformed to HyA_L. The collected HyA_L can be injected to form a line or letters or patterns using a syringe with a needle of 0.84 mm in diameter.

Measure the Reduction of Size, Volume, and Water Content and the Release of Fe³⁺ Ions During the Phase Transition from HyA_M to HyA_H

HyA_M with a diameter of 13 mm and a thickness of 2 mm was immersed in the DI water for up to 96 hours at room temperature and pressure. After immersed in DI water for 0.25 h, 0.5 h, 1 h, 1.5 h, 2.5 h, 4 h, 12 h, and 24 h, 36 h, 48 h, and 96 h, the gels were collected from the DI water using a tweezer and were then photographed respectively. The diameter of the sample was measured before and after immersion in DI water for a prescribed period, based on the scale bar in each photograph using the tools in ImageJ software. The mass and buoyant mass of the sample was measured before and after immersion in DI water for a prescribed period using the analytical balance (ME-T, Meter Toledo) and buoyant balance (analytical balance equipped with a density Kit, XPR-S, Meter Toledo). The volume (v) of the hydrogel before and after immersion in DI water for a prescribed period was calculated as:

$$v = \frac{{m}_{1} - {m}_{2}}{ρ} \qquad ( 6 )$$

where v is the volume of the hydrogel at a specific timepoint, m₁ and m₂ are the mass and buoyant mass of the hydrogel at a specific time point, and ρ is the density of water (i.e. 1 g/cm³).

The volume change ratio at a specific time point was calculated as:

$$Volume change ratio = \frac{v}{{v}_{0}} \qquad ( 7 )$$

where v is the volume of the hydrogel at that specific timepoint, and v0 is the volume of hydrogel before immersion in DI water.

After being immersed in DI water for 96 h, the crosslinked HyA hydrogel was lyophilized using a lyophilizer at the temperature of −54° C. and the pressure of 0.01 mBar. (FreeZone Benchtop Freeze Dryer, Labconco). The water content in the sample at a specific time point was calculated as:

$$Water content = ( \frac{m - {m}_{0}}{m} ) \times 100 \% \qquad ( 8 )$$

where m is the wet mass of the hydrogel at the prescribed timepoint, and m0 is the sample dry mass. The sample wet mass was measured after the gel was collected from the DI water and dried by a gentle wipe. The sample dry mass reached a constant after lyophilization and was then weighed as m0. The water content describes the mass percentage of water in the wet swelling hydrogel.

The Fe³⁺ ion release during the phase transition from HyA_M to HyA_H in DI water was measured using inductively coupled plasma-optical emission spectrometry (ICP-OES; Optima 8000, PerkinElmer, Waltham, Mass.). Specifically, HyA_M with a diameter of 13 mm and a thickness of 2 mm was immersed in 50 mL DI water in a 50 mL tube at room temperature and pressure. At the timepoints of 0 hour, 0.25 hour, 0.5 hour, 1 hour, 1.5 hours, 2.5 hours, 4 hours, 12 hours, 24 hours, 36 hours, 48 hours, and 96 hours, the immersion solution of 0.1 mL was collected from the tube at each timepoint and was diluted with a factor of 1:100 using DI water (Millipore). The diluted solution was loaded onto the autosampler for ICP-OES. The Fe³⁺concentration in the immersion solution was calculated based on the curves of Fe³⁺ standards at the respective concentrations of 0.1, 0.5, 1.0 mg/L, which was prepared by dissolving FeCl₃ in DI water. The cumulative amount of Fe³⁺ ion released from HyA hydrogel into the DI water at each timepoint was calculated using the following equation:

Fe³⁺ion released=c×v (9)

where c is the Fe³⁺ concentration in the immersion solution measured using ICP-OES, v is the volume of the total immersion solution.

The measurements of sample diameter, volume change ratio, water content, and Fe³⁺ ion release was performed in triplicate samples.

### Characterize Microstructure and Properties of the HyA_L, HyA_M, HyA_H, and HyA

Characterize the Microstructure and the Porosity of HyA_M, HyA_H, and HyA

The cross-sections of lyophilized HyA_M, HyA_H, and HyA were characterized using scanning electronic microscopy (SEM, Nova NanoSEM 450, FEI Co) with an accelerating voltage of 5 kV and a spot size of 3, at the magnification of 1000× and 10000×. The porosity of HyA_M, HyA_H, and HyA was determined in the SEM images using the image analysis tools in ImageJ software.

Characterize the Thermal Properties of HyA_L, HyA_M, HyA_H, and HyA

The HyA samples of interest were analyzed using a thermogravimetric analyzer (TGA; TG 209 F1 Libra®, Netzsch). For TGA, the lyophilized HyA_L, HyA_M, HyA_H, and HyA sample of 10 mg each were placed in alumina crucibles and heated from 30° C. to 800° C. at a heating rate of 10° C./min in a nitrogen (N₂) atmosphere with a N₂ flow rate of 20 mL/min. The sample mass change over the temperature was analyzed and plotted. The thermal properties of the samples were analyzed using a differential scanning calorimeter (DSC 214, Netzsch). For DSC measurements, the lyophilized HyA_L, HyA_M, HyA_H, and HyA sample of 5 mg each were placed in aluminum containers and an empty container was used as a reference. The samples were heated from 0° C. to 120° C., then cooled to 0° C., and reheated to 350° C. The heating and cooling rates were set as 10° C./min and the test was performed in a N₂ atmosphere with a N₂ flow rate of 10 mL/min.

Characterize the Chemical Bonding of HyA_L, HyA_M, HyA_H, and HyA

Fourier transform infrared spectroscopy-attenuated total reflection (FTIR-ATR, Nicolet iS10, ThermoFisher Scientific) was used to measure the transmittance of the HyA_L, HyA_M, HyA_H, and 5 w/v % HyA solution at the wavenumber of 4000-500 cm¹. Briefly, the sample was placed on the sample holder to cover the ATR crystal, a metallic cap was then placed above the sample to prevent water evaporation. FTIR-ATR measurement was performed using the absorbance mode with 64 scans. After the measurement for each sample, the sample holder and the metallic cap were cleaned using DI water and then dried using a cotton swab to avoid cross-contamination between the samples.

Perform the Tensile Testing for HyA_M and HyA_H

The tensile properties of HyA_M and HyA_H samples were tested using an Instron 5969 dual column testing system equipped with a 10 N load cell (Instron, Norwood, Mass.). The HyA_M sample was cut into a dimension of 50×10×2 mm, and the HyA_H sample was cut into a dimension of 20×4×0.5 mm for tensile testing. The tensile testing was performed with a 0.005 N preload and a crosshead speed of 10 mm/min. The tensile stress-strain curves were plotted from the data calculated based the load and extension. The elastic modulus was determined from the linear region of stress-strain curve by fitting a straight line between 0% and 20% strain. The percent of sample elongation at the breakage point was also calculated

Measure the Dynamic Modulus and Viscosity of HyA_L and HyA

The rheological properties of HyA_L and HyA (5 w/v %) were determined using a rheometer (MCR 92 with PP25 measuring system, Anton Paar). For this purpose, the HyA_L was prepared by injecting 5 w/v % HyA solution to 300 mM FeCl₃ solution using a syringe with a needle size of 0.35 mm and reacted for 4 hours. The storage modulus and loss modulus of the sample at the shear strain of 1%˜1000% (or shear stain of 0.01-10) was recorded at 25° C., similarly as described above. For viscosity measurement, the gap between the bottom plane of the spindle and stage was set as 1 mm, and the viscosity of the sample at the shear rate of 0.1 l/s˜100 l/s was recorded at 25° C.

### Determine the Conditions Required for 3D Printable HyA (HyA_P) and Characterization of 3D-Printed HyA

Determine the Conditions for Preparing HyA_P

Crosslinked HyA hydrogels were prepared by injecting 5 w/v % HyA solution into 300 mM FeCl₃ solution using a syringe with a needle size of 0.35 mm. The reaction time was set to be 5 minutes, 15 minutes, 30 minutes, and 120 minutes. The samples were collected using a spoon for rheological testing using the same method as described above. The storage modulus, loss modulus, and viscosity of crosslinked HyA hydrogels with different reaction time and HyA control (5 w/v %) were measured to highlight the conditions to achieve 3D-printable HyA (referred to as HyA_P).

Demonstrate Cold-Stage Method and Direct Writing Method for 3D-Printing of the HyA_P

To prepare the HyA_P, 10 mL of HyA solution at the concentration of 5 w/v % was injected to 40 mL of 300 mM FeCl₃ solution using a syringe with a needle size of 0.35 mm. After 15 minutes of reaction, the HyA_P was collected using a spoon and filled in the printing tube. HyA_P was printed onto the cold petri dish surface at 0° C. using by a 3D Bioplotter (Developer series, EnvisionTec, Germany). The architecture was changed by printing filaments with 0 and 90 angles between two successive layers. The injection pressure, speed of the printing head, nozzle size, and the distance between neighboring filaments was set as 1.5-3.0 bar, 5-10 mm/s, 0.84 mm, and 2.3 mm, respectively. After printing, the as-printed gel was immersed in DI water for 24 hour, and optical images of both top and bottom view of the printed sample were recorded using an optical microscope (SE303R-P, Amscope).

HyA_P was prepared using the same protocol described above, but the reaction time was extended to 1 hour. The HyA_P was directly printed onto a petri dish (10 cm in diameter) containing DI water. In order to increase the adhesion of the printed gel onto the substrate, a masking tape was placed on the bottom of dish to increase the roughness. The injection pressure, speed of the printing head, nozzle size, and the distance between two neighbor filaments was set as 1-1.5 bar, 3-8 mm/s, 0.84 mm, and 2.3 mm, respectively. After printing, the printed gel was immersed in DI water for 24 hours, and optical images of both top and bottom views of the printed sample were recorded by optical microscopy (SE303R-P, Amscope).

Evaluate the Morphology and Structural Stability of the Filaments Printed by Two Different Methods

For the cold-stage method group, a single filament was printed on a cold petri dish surface at 0° C. The printed filaments were immersed in DI water for 24 hours after they were kept at room temperature and pressure for 0 minutes, 1 minute, 5 minutes, and 10 minutes, respectively. For direct writing method group, a single filament was directly printed on a petri dish with DI water, and these filaments were continuously immersed in DI water during printing and after printing for 24 hours. The filaments were cut, and their cross-sections were imaged using optical microscopy (SE303R-P, Amscope). The height (H) and width (W) of the filament cross-section in the optical micrographs were measured using Image J. The aspect ratio (AR) of height/width was calculated for the filament cross-section. These measurements were repeated for 5 samples.

Evaluate the Cytocompatibility of HyA Hydrogels with Bone Marrow-Derived Stem Cells (BMSCs) In Vitro

Prepare HyA with Various Crosslinking Densities and Controls for Cell Culture

HyA powders were first disinfected under ultraviolet (UV) radiation for 1 hour. DI water was sterilized by autoclaving. To prepare the 300 mM FeCl₃ solution, FeCl₃ (#169430010, Sigma-Aldrich, St. Louis, Mo.) was dehydrated in an oven at 120° C. for 30 minutes. Afterward, the FeCl₃ was weighed while it was still hot and then dissolved in the sterilized DI water. HyA hydrogel samples of HyA_H_3D, HyA_H, HyA_M, HyA_P, and HyA_L were prepared using the similar method as described in Sections 2.2 and 2.5. Specifically, to get HyA_M, 5 w/v % HyA solution was injected onto a petri dish to form a thin layer using a syringe with a 0.5 mm needle. The thin layer of HyA was then crosslinked by adding 300 mM FeCl₃ solution for 3 min. The as-prepared HyA_M film had a thickness of 0.47+0.06 mm and was punched to a cylindrical shape with a diameter of 12.17+0.15 mm.

To prepare HyA_H, the prepared HyA_M cylinders were immersed in sterilized DI water for 24 hours. The prepared HyA_H cylinders had a diameter of 5.43+0.12 mm and a thickness of 0.23±0.06 mm. To prepare HyA_P and HyA_L, 5 w/v % HyA solution was injected into the 300 mM FeCl₃ solution using a syringe with a 0.35 mm needle. The reaction times were 30 minutes for HyA_P and 1 hour for HyA_L. For cell study, the as prepared HyA_P and HyA_L were manually to printed onto the respective sterilized glass slides and form a hydrogel layer to cover the whole area of the glass slide. The thickness of the gel was measured to be 0.63±0.15 mm for HyA_P and 0.47±0.06 mm for HyA_L. To prepare HyA_H_3D, the as-prepared HyA_P was manually printed onto a petri dish using a syringe with a 0.5 mm needle. The filaments in the first layer were printed in parallel and the orientation was defined as the 0° angle. The filaments in the second layer were printed in parallel with an orientation of 90° angle to the first layer. The distance between the parallel filaments in both layers was 1.1±0.34 mm. The printed HyA_P of two layers was immersed in DI water for 24 hours and cut to a cylindrical shape with a diameter of 5.6+0.17 mm. The thickness of HyA_3D sample was 0.43±0.15 mm.

HyA and Fe³⁺ ion groups were included as control groups in the cell study. Specifically, 3 mg of HyA powders (equivalent to the amount of HyA in a single HyA hydrogel sample) was added into each well, serving as the HyA control. The cells were cultured in 3 mL media in each well, and thus the HyA control had a concentration of 1 mg/mL HyA in the media. The Fe³⁺ ion control groups included two different Fe³⁺ ion concentrations to represent the low and high Fe³⁺ ion concentrations in crosslinked HyA hydrogels. The upper concentration of Fe³⁺ ions was defined as the theoretical concentration of Fe³⁺ ions in the media when the Fe³⁺ ions in a HyA_L sample were completely released and dissolved; while the lower concentration of Fe³⁺ ions was determined as the theoretical concentration of Fe³⁺ ions in the media when the Fe³⁺ ions in a HyA_H sample were completely released and dissolved.

To measure the amount of Fe³⁺ ions in HyA_L and HyA_H samples, we dissolved the HyA_L and HyA_H samples in the 10 mL 2 wt. % nitride acid, respectively. The 2 wt. % nitride acid was used as the solvent because it serves as a cleaning agent for ICP-OES and it can completely dissolve the crosslinked hydrogels to release all the Fe³⁺ ions into the solution. The nitride acid solutions containing Fe³⁺ ions were then diluted with DI water at a dilution factor of 1:500. The Fe³⁺ concentrations in the nitride acid solution were measured using ICP-OES, similarly as described in Section 2.3. The control group with the higher concentration of 6.08 mM Fe³⁺ ions (referred to as Fe@6.08 mM group) in the media was used to serve as Fe³⁺ control for HyA_L, and the group with a lower concentration of 2.09 mM Fe³⁺ ions (referred to as Fe@2.09 mM group) in the media was used to serve as Fe³⁺ ion control for HyA_H.

### Demonstrate the Cytocompatibility of HyA Hydrogels in Bone Marrow Mesenchymal Stem Cell (BMSC) Culture

Following the protocol approved by the Institutional Animal Care and Use Committee (IACUC) at the University of California at Riverside (UCR), rat BMSCs were harvested and cultured as described by Rutherford, D., et al., Journal of Biomedical Materials Research Part B: Applied Biomaterials 2020, 108 (3), 925-938; and Zhang, C., et al., ACS Biomaterials Science & Engineering 2019, 6 (1), 517-538. Briefly, the distal and proximal ends of the femoral and tibial bones were dissected, and the bone marrow was flushed out of the bone cavity by Dulbecco's Modified Eagle Media (DMEM, #SLBC9050, high glucose, D5648, Sigma-Aldrich, St. Louis, Mo.) supplemented with 10% fetal bovine serum (FBS, HyClone, #SH30910, Thermo Fisher Scientific Inc., Waltham, Mass.) and 1% penicillin/streptomycin (P/S, HyClone, #SV30010, Thermo Fisher Scientific, Inc., Waltham, Mass.) using a syringe and collected in the centrifuge tube. The collected cells were filtered using a 70-μm nylon strainer (Fisher Scientific, NH, USA) and then cultured in media under standard cell culture conditions (i.e., 37° C., 5%/95% CO2/air, humidified, sterile environment) to 90-95% confluency. The descriptions for cell harvesting were reprinted (adapted) with permission from (Zhang, C.; Lin, J.; Nguyen, N.-Y. T.; Guo, Y.; Xu, C.; Seo, C.; Villafana, E.; Jimenez, H.; Chai, Y.; Guan, R. Antimicrobial Bioresorbable Mg—Zn—Ca Alloy for Bone Repair in a Comparison Study with Mg—Zn—Sr Alloy and Pure Mg. ACS Biomaterials Science & Engineering 2019, 6 (1), 517-538). Copyright (2020) American Chemical Society.

Cytocompatibility of HyA hydrogels with BMSCs was evaluated using the direct exposure method, as described in the previous studies.⁴¹ Briefly, BMSCs were seeded to 12-well plates with a seeding density of 10,000 cells/cm². The cells in each well were cultured in 3 mL media under a standard environment (i.e., 37° C., 5%/95% CO₂/air, humidified, sterile environment) in an incubator for 24 hours to form a monolayer of cells. After the prescribed cell culture, the cells were imaged at the bright field using a fluorescence microscope (Eclipse Ti and NIS software, Nikon, Melville, N.Y., USA). Afterward, the media and non-adhered cells were removed from each well. The cells were rinsed using Phosphate Buffer solution (PBS) three times and 3 mL fresh media was added into each well expect HyA and Fe³⁺ control groups.

For HyA and Fe³⁺ control groups, 3 mL of as-prepared media containing 1 mg/L HyA, 2.09 mM Fe³⁺ ions, or 6.08 mM Fe³⁺ ions were added to each respective well. For all the samples of HyA_H_3D, HyA_H, HyA_M, HyA_P, and HyA_L, the samples were loaded in the respective well on top of the adhered cells. As mentioned earlier, the HyA_P and HyA_L were injected on the glass slides. When loading HyA_P and HyA_L in the well plate, the side with gel was faced down to direct contact with the cells. For other hydrogel groups, a glass slide was placed in the well-plate on top of each sample. Glass control, Cell control (without samples), and Media control (without cells and samples) were also included in the study. Upon adding the samples, the pH values of the media were measured using a precalibrated pH meter (Symphony, Model SB70P, VWR). The BMSCs were then cultured with the samples for 24 hours under standard cell culture conditions (i.e., 37° C., 5%/95% CO2/air, humidified, sterile environment).

After the prescribed cell culture, glass slides were removed from each well, and the post-culture media were collected for further analysis. BMSCs attached on well-plates were rinsed by PBS three times and fixed with 4% formaldehyde (10% neutral buffered formalin; VWR, Radnor, Pa., USA) for 20 min. The fixed BMSCs were stained with Alexa Flour 488-phalloidin (A12379, Life technologies) for F-actin for 20 min and 4′,6-diamidino-2-phenylindole dilactate (DAPI, Invitrogen) for nuclei for 5 min. BMSCs directly in contact with the sample (i.e., direct contact) and surrounding each sample (i.e., indirect contact) were imaged using a fluorescence microscope (Eclipse Ti and NIS software, Nikon, Melville, N.Y., USA). The optical images of cells under direct and indirect contact conditions were also obtained using the bright field of the above fluorescence microscope. DAPI-stained nuclei were counted to determine cell adhesion density per unit area. At least five fluorescence images of BMSCs under direct contact conditions and five fluorescence images of BMSCs under indirect contact conditions were used for cell counting and statistical analyses of the data.

Demonstrate HyA Degradation and Associated Changes in Culture Media Via Media Analysis of Degradation Products, pH Value, and Concentrations of Fe³⁺ and Ca²⁺ Ions

The photographs of hydrogels loaded in the media in the well-plate were imaged before and after cell culture. Media collected in the 15 mL centrifuge tubes were also photographed to identify the degradation products of the hydrogels. The pH values of post-culture media were measured using a precalibrated pH meter (Symphony, Model SB70P, VWR) immediately after collection. Fe³⁺ and Ca²⁺ ions in the post-culture media were measured using ICP-OES as described above. Before the measurement, the media samples were centrifuged at 5000 revolutions per minute (RPM) for 3 minutes to separate the solid. The supernatant was collected and diluted by DI water for the measurement. The dilution factor of the media sample for measuring Fe³⁺ concentration is 1:3 for the groups of HyA, Glass, Cell, and Media, and 1:100 for other groups. The dilution factor of the media sample for measuring Ca²⁺ ions was 1:100 for all the samples.

The hydrogels described herein allow for tunable crosslinking and reversible phase transition by controlling concentrations of Fe³⁺ and H⁺ ions as well as the reaction time, which advanced from a single state to multiple reversible states. Second, equations described herein demonstrate the relationships of [Fe³⁺]/[H⁺] ratio and HyA concentration with the crosslinking density; these equations can be applied to other materials crosslinked via carboxylate-Fe³⁺ coordination. Third, a mechanism of creating 3D printable hydrogel inks utilizing reversible phase transition via metal-ligand coordination is provided and successfully applied to 3D printing of the hydrogel inks using two different methods. The 3D printing methods and mechanisms of creating printable hydrogel inks can be applied to a wide range of other materials containing carboxyl groups.

### Additional Data

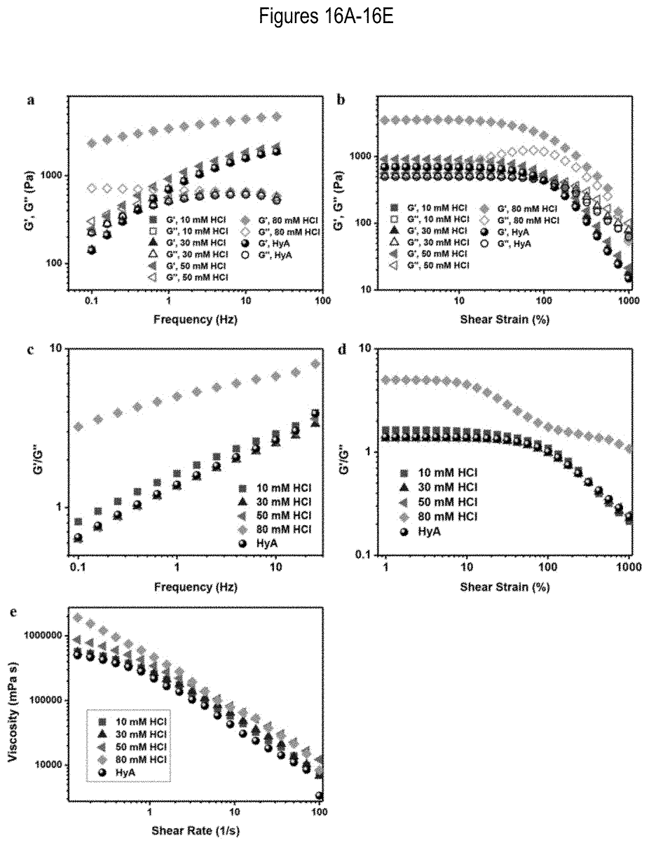

Additional data is provided in FIGS. 16-23.

FIG. 16 shows the rheological results of 5 w/v % HyA gels at different HCl concentrations of 10, 30, 50, and 80 mM. As shown in FIG. 16 a, when the HCl concentration were 10-50 mM, storage modulus (G′) of the sample was lower than the corresponding loss modulus (G″) at the low frequency of 0.1 Hz, and G′ was greater than the corresponding G″ at the frequency of >0.3 Hz; when the HCl concentration was 80 mM, the G′ was greater than the corresponding G″ at the frequency of 0.1-30 Hz, showing a characteristic of solid material. As shown in FIG. 16 b, samples with HCl concentrations of 10-50 mM exhibited that G′ was greater than the corresponding G″ at the shear strain range of 1%-100% and G′ was lower than the corresponding G″ at the high shear strain of >200%; while the sample with an HCl concentration of 80 mM showed that G′ was greater than the corresponding G″ at the shear strain of 1%-1000%. All the samples showed a shear-thinning property. FIGS. 16c and d show the ratios of G′/G″ at different frequencies and shear strains, respectively. The structural stability of the HyA sample was higher than the control group when the HCl concentration was 10 mM, while the samples had reduced structural stability when the HCl concentration was 30-50 mM. When the HCl concentration was 80 mM, the HyA had the best structural stability. As shown in FIG. 16e, all the samples had a higher viscosity than the control group, and the viscosity was increased when increasing the HCl concentration at the shear rate of 0.1-2 s⁻¹.

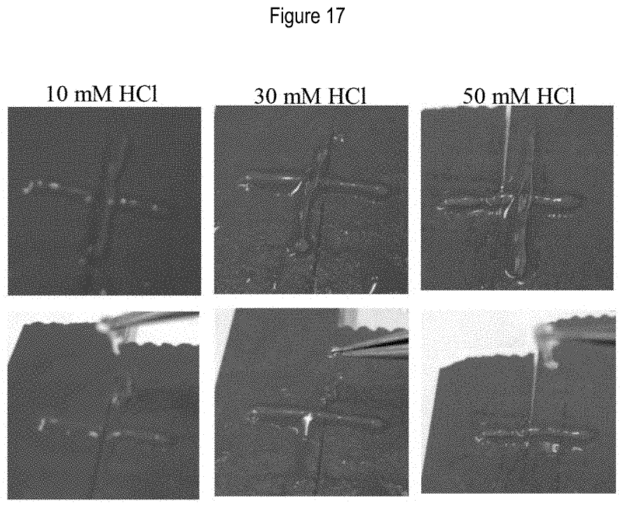

To determine the adhesion between the neighboring layers during the direct writing of HyA solutions into the FeCl₃ solution, FIG. 17 shows the photographs indicating the adhesion between the crossed hydrogel filaments. The crossed hydrogel filaments were prepared by injecting 5 w/v % HyA solution with 10-50 mM HCl concentrations into 30 mM FeCl₃ solution and reacting for 10 s. When the HCl concentrations of the HyA solution were 10-50 mM, the top filament was easily peeled off from the bottom filament using a tweezer, which indicated that HyA solutions at 10-50 mM HCl concentrations are not able to form a stable 3D structure when being printed in the FeCl₃ solution.

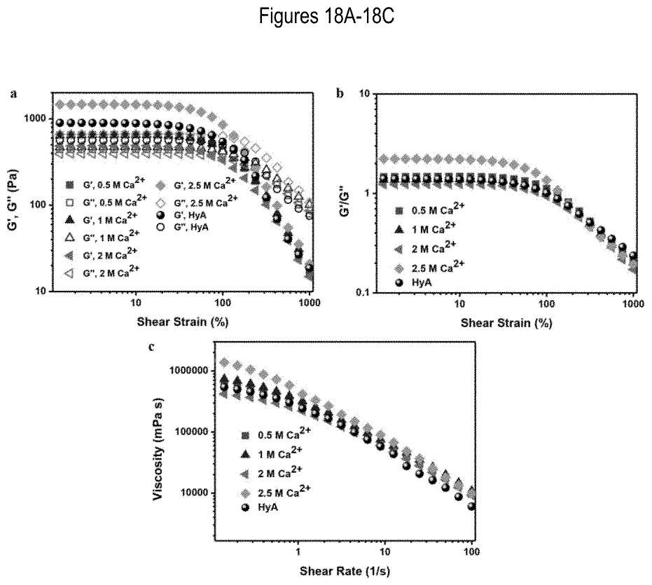

FIG. 18 shows the rheological results of 5 w/v % HyA solutions at different Ca²⁺ concentrations of 0.5, 1, 2, and 2.5 M. As shown in FIG. 18 a, all the samples exhibited that G′ was greater than the corresponding G″ at the shear strain range of %-100% and G′ was lower than the corresponding G″ at the high shear strain of >100%, indicating a shear-thinning property.

FIG. 18b shows the ratios of G′/G″ at different shear strains. At the shear strain of 1%-100%, HyA solutions at the Ca²⁺ concentrations of 0.5 and 1 M had similar structural stability to HyA controls, while the HyA solution at the Ca²⁺ concentration of 2 M showed a decreased structural stability. When the Ca²⁺ concentration was 2.5 M, the structural stability of the HyA solution was dramatically increased at the shear strain of 1%-100%. As shown in FIG. 16c, at the shear rate of 0.1-2 s⁻¹, HyA solutions at the Ca²⁺ concentrations of 2 M had a lower viscosity than HyA control while all other groups showed a greater viscosity.

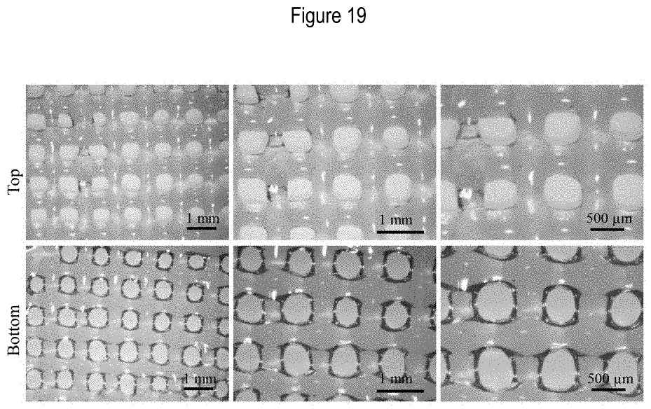

FIG. 19 shows the optical micrographs of crosslinked HyA hydrogels that were printed on cold stage and then immersed in 50 mM FeCl₃ solution followed by immersing in DI water for 24 hours, with both top view (Top) and bottom view (Bottom) of the 3D-printed hydrogels. The HyA_P was 5 w/v % HyA solutions with 2.5 M Ca²⁺. As shown in FIG. 19, the pores of the 3D constructs changed from a designed square to a round shape and the printed layers had merged into one layer, which indicated that the HyA solution at a Ca²⁺ concentration of 2.5 M cannot provide enough structural stability for the 3D printing.

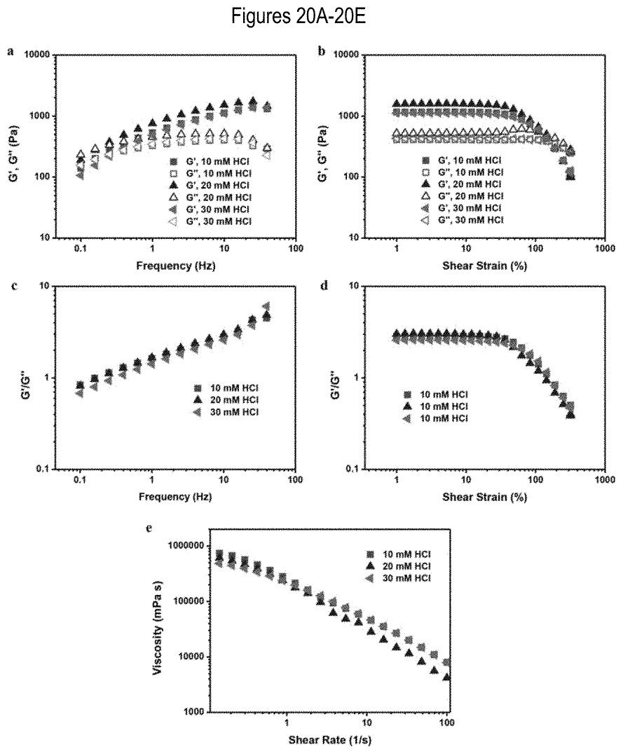

FIG. 20 shows the rheological results of 5 w/v % HyA solutions at 2 M Ca²⁺ and different HCl concentrations of 10, 20, and 30 mM. As shown in FIG. 20 a, all the samples had their G′ lower than the corresponding G″ at the low frequency of 0.1 Hz and G′ greater than the corresponding G″ at the frequency of >1 Hz, showing a characteristic of liquid material at low-frequency range and property of solid material at high-frequency range. As shown in FIG. 20 b, all the samples exhibited that G′ was greater than the corresponding G″ at the shear strain range of 1%-100% and G′ was lower than the corresponding G″ at the high shear strain of >200%, suggesting a shear-thinning property.

FIGS. 20c and d show the ratios of G′/G″ at different frequencies and shear strains, respectively. The structural stability of the HyA samples at HCl concentrations of 10 and 20 mM was similar but higher than the sample at an HCl concentration of 30 mM, at the frequency of 0.1-10 Hz and shear strain of 1%-40/o. As shown in FIG. 20e, the HyA solution had reduced viscosity when increasing the HCl concentration at the low shear rate of 0.1-1 s⁻¹. At the shear strain of 2-100 Hz, HyA solution at the HCl concentrations of 10 and 30 mM showed similar viscosity which is greater than that of HyA solution at an HCl concentration of 20 mM.

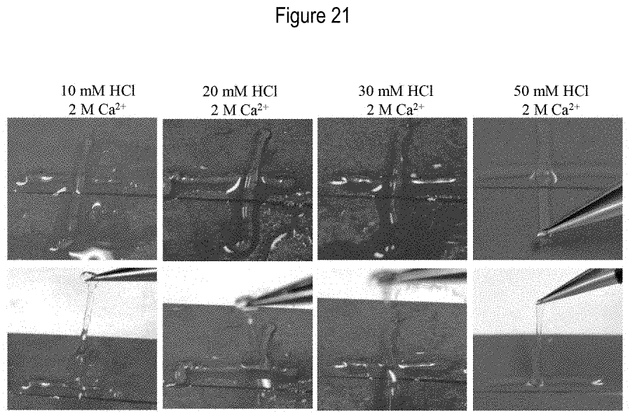

FIG. 21 shows the photographs indicating the adhesion between the crossed hydrogel filaments. The crossed hydrogel filaments were prepared by injecting 5 w/v % HyA solution at 2 M Ca²⁺ content and different HCl concentrations of 10-30 mM HCl in 30 mM FeCl₃ solution and reacting for 10 s. When the HCl concentration of the HyA solution was 10 mM, the top filament was easily peeled off from the bottom filament using a tweezer, which indicated that HyA solutions with 10 mM HCl concentration and 2 M Ca²⁺ are not able to form a stable 3D structure when being printed in the FeCl₃ solution. When the HCl concentrations of the HyA solution were 20-50 mM, the top filaments stuck on the bottom filaments, which indicated that these HyA solutions can form a stable 3D structure when being printed in the FeCl₃ solution.

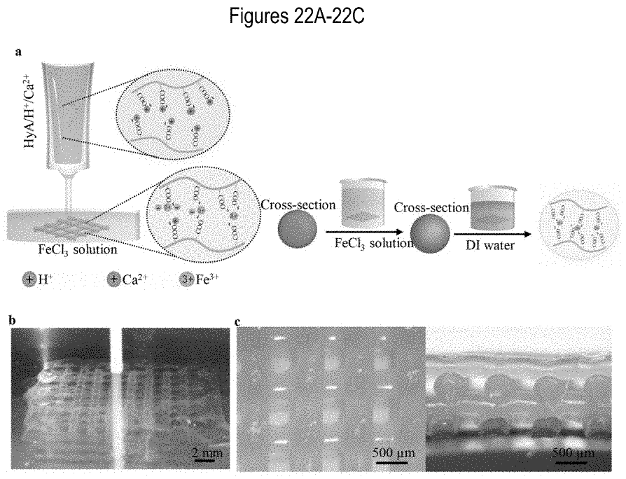

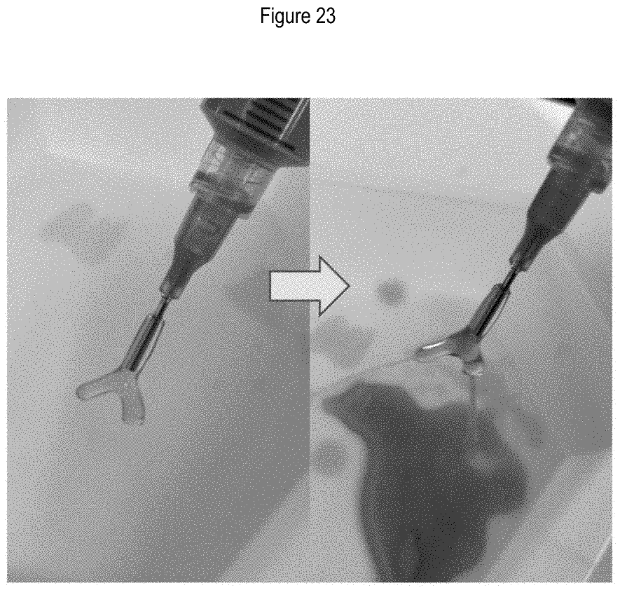

FIG. 22 demonstrated the direct writing of HyA hydrogels in FeCl₃ solution. FIG. 22a shows the schematic diagram of direct writing of HyA hydrogels in FeCl₃ solution. Specifically, HyA solution added with H⁺ and Ca²⁺ ions were firstly directly printed into 30 mM FeCl₃ solution, the printed construct was then immersed in 50 mM FeCl₃ solution for 3 min, followed by immersed in DI water for 24 hours. As shown in FIG. 22b, a stable 3D construct was printed via direct writing of HyA solution in 30 mM FeCl₃ solution. FIG. 22c shows the optical micrographs of crosslinked HyA hydrogels prepared via direct writing of HyA solution in FeCl₃ solution. The HyA solution with 30 mM HCl and 2 M Ca²⁺ were printed into 30 mM FeCl₃ solution and then immersed in 50 mM FeCl₃ solution followed by immersed in DI water for 24 hours. Both the top view (Left) and cross-sectional view (Right) of the images show that the 3D-printed hydrogels had a 3D layer-by-layer as designed.

FIG. 23 demonstrates the tubular structure of the hydrogel filaments. As shown in the photograph, the needle (40.84 mm) of the syringe was inserted into the tubular hydrogel filaments, the cell culture media (red) was then injected into the hydrogel filament and discharged from the two outlets. To prepare the tubular hydrogel filaments, 5 w/v % HyA solution was injected onto the petri dish to form the “Y-shaped” hydrogel filaments using a syringe with a (1.5 mm needle. The as-injected HyA solution was immersed in 50 mM FeCl₃ solution for 20 s followed by immersed in DI water for 3 min. The tubular hydrogel filaments with crosslinked shell (solid) and non-crosslinked core (liquid) was then obtained.

Hydrogels of the invention are 3D printable due to the dynamic coordination of their innate carboxylic groups and metal ions. The hydrogels are compatible with all extrusion-based 3D printers, such as 3D-Bioplotters from EnvisionTEC, bioprinters from CELLINK, bioprinters from Allegro 3D, 3D printers from REGENHU, and bioprinters from Allevi. More importantly, adding methyl acrylate or other functional groups is not required; the hydrogels are less toxic. Since functionalization of the hydrogels is not required, preparation of the hydrogels is less process-intensive and more cost-effective. Additionally, a UV module is not needed for 3D printers to print these hydrogels.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A hydrogel having tunable crosslinking density and reversible phase transition that is suitable as an ink for three-dimensional (3D) printing.

2. The hydrogel of claim 1 that comprises a polymer that comprises carboxyl groups.

3. The hydrogel of claim 2, wherein the polymer is poly (acrylamide-co-acrylic acid), Poly (acrylic acid), carboxylated gelatin, carboxylated cellulose, carboxymethyl cellulose, or chondroitin sulfate.

4. The hydrogel of claim 1, wherein the polymer is hyaluronic acid, a salt of it, or its derivatives having carboxyl groups.

5. The hydrogel of claim 2, wherein the carboxyl groups are coordinated with metal ions.

6. The hydrogel of claim 5, wherein the metal ions are in a monodentate coordination state.

7. The hydrogel of claim 5, wherein the metal ions are in a bidentate coordination state.

8. The hydrogel of claim 5, wherein the metal ions are in a tridentate coordination state.

9. The hydrogel of claim 2, wherein the hydrogel further comprises hydrogen ions bonded to the carboxyl groups.

10. The hydrogel of claim 1, that comprises one or more metal ions selected from the group consisting of Fe³⁺, Al⁺, Sc³⁺, Cr³⁺, Ga³⁺, In³⁺, Ce⁴⁺, V³⁺, V²⁺, Hg²⁺, Pb²⁺, Mn²⁺, Be²⁺, Co²⁺, Mg²⁺, Ca²⁺, Sr²⁺, Sn²⁺, Ba²⁺, Cu²⁺, Zn²⁺, Cd²⁺, Ni²⁺, and Fe²⁺.

11. The hydrogel of claim 1, that comprises Fe³⁺ metal ions.

12. The hydrogel of claim 4, wherein the hyaluronic acid has a molecular weight in the range of from about 5,000 Da to about 20,000,000 Da.

13. The hydrogel of claim 4, wherein the hyaluronic acid has a molecular weight in the range of from about 500,000 Da to about 8,000,000 Da.

14. The hydrogel of claim 2, wherein at most 100% of the carboxyl groups are associated with a metal ion.

15. The hydrogel of claim 2, wherein at most 100% of the carboxyl groups are associated with a hydrogen ion.

16. An 3D printable ink that comprises a hydrogel as described in claim 1.

17. The 3D printable aqueous ink of claim 16 that has a storage and loss modulus of 0.1-1,000,000 Pa at room temperature.

18. The 3D printable aqueous ink of claim 16, that has shear-thinning properties.

19. The 3D printable aqueous ink of claim 16, that has a viscosity of ≥1 mPa·s at room temperature.

20. A method for 3D printing a three-dimensional object, the method comprising, providing a 3D ink as described in claim 16; and 3D printing the three-dimensional object using the 3D ink as a feedstock.

21. The method of claim 20, wherein 3D printing the three-dimensional object comprises extruding the 3D ink into a pattern that forms the three-dimensional object on a cold stage.

22. The method of claim 20, wherein 3D printing the three-dimensional object comprises: extruding the 3D ink into a pattern that forms the three-dimensional object through direct printing in aqueous solution with a pH of 2-13 or in pure water.

23. The method of claim 20, wherein 3D printing the three-dimensional object comprises: extruding the 3D ink into a pattern that forms the three-dimensional object through direct printing in aqueous solution with metal ions.

24. The method of claim 20, wherein crosslinking of the hydrogel or reversible phase transition of the hydrogel is achieved by controlling a) the ratio of metal ions to hydrogen ions in the hydrogel, b) the concentration of the polymer in the hydrogel, or c) reaction time.

25. The method of claim 20, wherein crosslinking of the hydrogel or reversible phase transition of the hydrogel is achieved by controlling a) the ratio of metal ions to hydrogen ions in the hydrogel, b) the concentration of the polymer in the hydrogel, and c) reaction time.

26. The method of claim 20, wherein the aqueous solution comprises one or more metal ions selected from the group consisting of Fe³⁺, Al³⁺, Sc³⁺, Cr³⁺, Ga³⁺, In³⁺, Ce⁴⁺, V³⁺, V²⁺, Hg²⁺, Pb²⁺, Mn²⁺, Be²⁺, Co²⁺, Mg²⁺, Ca²⁺, Sr²⁺, Sn²⁺, Ba²⁺, Cu²⁺, Zn²⁺, Cd²⁺, Ni²⁺, and Fe²⁺.

27. The method of claim 20, wherein the three-dimensional object has solid or tubular hydrogel filaments.

28. The method of claim 20, wherein the three-dimensional object has solid and tubular hydrogel filaments.

29. The method of claim 20, further comprising packaging the 3D ink in a 3D printer cartridge.

30. The method of claim 20, wherein the three-dimensional object is a wound healing device.

31. The method of claim 20, wherein the three-dimensional object is a scaffold for growing cartilage.

32. The method of claim 20, wherein the three-dimensional object is an aneurysm treatment device.

33. The method of claim 20, wherein the three-dimensional object is a scaffold for tissue regeneration.

34. A wound healing device that comprises a hydrogel as described in claim 1.

35. A scaffold for cartilage that comprises a hydrogel as described in claim 1.

36. An aneurysm treatment device that comprises a hydrogel as described in claim 1.

37. A scaffold for tissue regeneration that comprises a hydrogel as described in claim 1.

38. A method for preparing a 3D printable ink comprising, combining hyaluronic acid and Fe³⁺ ions and adjusting the concentration of Fe³⁺ or H⁺ to provide the 3D printable ink.

39. The method of claim 38, wherein the 3D printable ink comprises a hydrogel that comprises hyaluronic acid and Fe³⁺ ions.
